SEQUENCE LISTING (137 sequences)

SEQ ID NO: 1 - protein, 15 aa, Artificial Sequence
  FOL-005, Peptide with similarity to sub-region of osteopontin
VDTYDGDISVVYGLR

SEQ ID NO: 2 - protein, 15 aa, Artificial Sequence
  Peptide with similarity to sub-region of osteopontin
VDTYDGDISVVYGLS

SEQ ID NO: 3 - protein, 14 aa, Artificial Sequence
  Peptide with similarity to sub-region of osteopontin
VDTYDGDISVVYGL

SEQ ID NO: 4 - protein, 14 aa, Artificial Sequence
  Peptide with similarity to sub-region of osteopontin
DTYDGDISVVYGLR

SEQ ID NO: 5 - protein, 14 aa, Artificial Sequence
  Peptide with similarity to sub-region of osteopontin
TYDGDISVVYGLRS

SEQ ID NO: 6 - protein, 13 aa, Artificial Sequence
  Peptide with similarity to sub-region of osteopontin
VDTYDGDISVVYG

SEQ ID NO: 7 - protein, 13 aa, Artificial Sequence
  Peptide with similarity to sub-region of osteopontin
DTYDGDISVVYGL

SEQ ID NO: 8 - protein, 13 aa, Artificial Sequence
  Peptide with similarity to sub-region of osteopontin
TYDGDISVVYGLR

SEQ ID NO: 9 - protein, 13 aa, Artificial Sequence
  Peptide with similarity to sub-region of osteopontin
YDGDISVVYGLRS

SEQ ID NO: 10 - protein, 12 aa, Artificial Sequence
  Peptide with similarity to sub-region of osteopontin
VDTYDGDISVVY

SEQ ID NO: 11 - protein, 12 aa, Artificial Sequence
  Peptide with similarity to sub-region of osteopontin
DTYDGDISVVYG

SEQ ID NO: 12 - protein, 12 aa, Artificial Sequence
  Peptide with similarity to sub-region of osteopontin
TYDGDISVVYGL

SEQ ID NO: 13 - protein, 12 aa, Artificial Sequence
  Peptide with similarity to sub-region of osteopontin
YDGDISVVYGLR

SEQ ID NO: 14 - protein, 12 aa, Artificial Sequence
  Peptide with similarity to sub-region of osteopontin
DGDISVVYGLRS

SEQ ID NO: 15 - protein, 11 aa, Artificial Sequence
  Peptide with similarity to sub-region of osteopontin
VDTYDGDISVV

SEQ ID NO: 16 - protein, 11 aa, Artificial Sequence
  Peptide with similarity to sub-region of osteopontin
DTYDGDISVVY

SEQ ID NO: 17 - protein, 11 aa, Artificial Sequence
  Peptide with similarity to sub-region of osteopontin
TYDGDISVVYG

SEQ ID NO: 18 - protein, 11 aa, Artificial Sequence
  Peptide with similarity to sub-region of osteopontin
YDGDISVVYGL

SEQ ID NO: 19 - protein, 11 aa, Artificial Sequence
  Peptide with similarity to sub-region of osteopontin
DGDISVVYGLR

SEQ ID NO: 20 - protein, 11 aa, Artificial Sequence
  Peptide with similarity to sub-region of osteopontin
GDISVVYGLRS

SEQ ID NO: 21 - protein, 10 aa, Artificial Sequence
  Peptide with similarity to sub-region of osteopontin
VDTYDGDISV

SEQ ID NO: 22 - protein, 10 aa, Artificial Sequence
  Peptide with similarity to sub-region of osteopontin
DTYDGDISVV

SEQ ID NO: 23 - protein, 10 aa, Artificial Sequence
  Peptide with similarity to sub-region of osteopontin
TYDGDISVVY

SEQ ID NO: 24 - protein, 10 aa, Artificial Sequence
  Peptide with similarity to sub-region of osteopontin
YDGDISVVYG

SEQ ID NO: 25 - protein, 10 aa, Artificial Sequence
  Peptide with similarity to sub-region of osteopontin
DGDISVVYGL

SEQ ID NO: 26 - protein, 10 aa, Artificial Sequence
  Peptide with similarity to sub-region of osteopontin
GDISVVYGLR

SEQ ID NO: 27 - protein, 10 aa, Artificial Sequence
  Peptide with similarity to sub-region of osteopontin
DISVVYGLRS

SEQ ID NO: 28 - protein, 9 aa, Artificial Sequence
  Peptide with similarity to sub-region of osteopontin
VDTYDGDIS

SEQ ID NO: 29 - protein, 9 aa, Artificial Sequence
  Peptide with similarity to sub-region of osteopontin
DTYDGDISV

SEQ ID NO: 30 - protein, 9 aa, Artificial Sequence
  Peptide with similarity to sub-region of osteopontin
TYDGDISVV

SEQ ID NO: 31 - protein, 9 aa, Artificial Sequence
  Peptide with similarity to sub-region of osteopontin
YDGDISVVY

SEQ ID NO: 32 - protein, 9 aa, Artificial Sequence
  Peptide with similarity to sub-region of osteopontin
DGDISVVYG

SEQ ID NO: 33 - protein, 9 aa, Artificial Sequence
  Peptide with similarity to sub-region of osteopontin
GDISVVYGL

SEQ ID NO: 34 - protein, 9 aa, Artificial Sequence
  Peptide with similarity to sub-region of osteopontin
DISVVYGLR

SEQ ID NO: 35 - protein, 9 aa, Artificial Sequence
  Peptide with similarity to sub-region of osteopontin
ISVVYGLRS

SEQ ID NO: 36 - protein, 8 aa, Artificial Sequence
  Peptide with similarity to sub-region of osteopontin
VDTYDGDI

SEQ ID NO: 37 - protein, 8 aa, Artificial Sequence
  Peptide with similarity to sub-region of osteopontin
DTYDGDIS

SEQ ID NO: 38 - protein, 8 aa, Artificial Sequence
  Peptide with similarity to sub-region of osteopontin
TYDGDISV

SEQ ID NO: 39 - protein, 8 aa, Artificial Sequence
  Peptide with similarity to sub-region of osteopontin
YDGDISVV

SEQ ID NO: 40 - protein, 8 aa, Artificial Sequence
  Peptide with similarity to sub-region of osteopontin
DGDISVVY

SEQ ID NO: 41 - protein, 8 aa, Artificial Sequence
  Peptide with similarity to sub-region of osteopontin
GDISVVYG

SEQ ID NO: 42 - protein, 8 aa, Artificial Sequence
  Peptide with similarity to sub-region of osteopontin
DISVVYGL

SEQ ID NO: 43 - protein, 8 aa, Artificial Sequence
  Peptide with similarity to sub-region of osteopontin
ISVVYGLR

SEQ ID NO: 44 - protein, 7 aa, Artificial Sequence
  Peptide with similarity to sub-region of osteopontin
VDTYDGD

SEQ ID NO: 45 - protein, 7 aa, Artificial Sequence
  Peptide with similarity to sub-region of osteopontin
DTYDGDI

SEQ ID NO: 46 - protein, 7 aa, Artificial Sequence
  Peptide with similarity to sub-region of osteopontin
TYDGDIS

SEQ ID NO: 47 - protein, 7 aa, Artificial Sequence
  Peptide with similarity to sub-region of osteopontin
YDGDISV

SEQ ID NO: 48 - protein, 7 aa, Artificial Sequence
  Peptide with similarity to sub-region of osteopontin
DGDISVV

SEQ ID NO: 49 - protein, 7 aa, Artificial Sequence
  Peptide with similarity to sub-region of osteopontin
GDISVVY

SEQ ID NO: 50 - protein, 7 aa, Artificial Sequence
  Peptide with similarity to sub-region of osteopontin
DISVVYG

SEQ ID NO: 51 - protein, 7 aa, Artificial Sequence
  Peptide with similarity to sub-region of osteopontin
ISVVYGL

SEQ ID NO: 52 - protein, 6 aa, Artificial Sequence
  Peptide with similarity to sub-region of osteopontin
DTYDGD

SEQ ID NO: 53 - protein, 6 aa, Artificial Sequence
  Peptide with similarity to sub-region of osteopontin
TYDGDI

SEQ ID NO: 54 - protein, 6 aa, Artificial Sequence
  Peptide with similarity to sub-region of osteopontin
YDGDIS

SEQ ID NO: 55 - protein, 6 aa, Artificial Sequence
  Peptide with similarity to sub-region of osteopontin
DGDISV

SEQ ID NO: 56 - protein, 6 aa, Artificial Sequence
  Peptide with similarity to sub-region of osteopontin
GDISVV

SEQ ID NO: 57 - protein, 6 aa, Artificial Sequence
  Peptide with similarity to sub-region of osteopontin
DISVVY

SEQ ID NO: 58 - protein, 6 aa, Artificial Sequence
  Peptide with similarity to sub-region of osteopontin
ISVVYG

SEQ ID NO: 59 - protein, 5 aa, Artificial Sequence
  Peptide with similarity to sub-region of osteopontin
TYDGD

SEQ ID NO: 60 - protein, 5 aa, Artificial Sequence
  Peptide with similarity to sub-region of osteopontin
YDGDI

SEQ ID NO: 61 - protein, 5 aa, Artificial Sequence
  Peptide with similarity to sub-region of osteopontin
DGDIS

SEQ ID NO: 62 - protein, 5 aa, Artificial Sequence
  Peptide with similarity to sub-region of osteopontin
GDISV

SEQ ID NO: 63 - protein, 5 aa, Artificial Sequence
  Peptide with similarity to sub-region of osteopontin
DISVV

SEQ ID NO: 64 - protein, 5 aa, Artificial Sequence
  Peptide with similarity to sub-region of osteopontin
ISVVY

SEQ ID NO: 65 - protein, 5 aa, Artificial Sequence
  Peptide with similarity to sub-region of osteopontin
SVVYG

SEQ ID NO: 66 - protein, 300 aa, Homo sapiens
  Osteopontin
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 67 - protein, 16 aa, Artificial Sequence
  Peptide with similarity to sub-region of osteopontin
VDTYDGRGDSVVYGLR

SEQ ID NO: 68 - protein, 17 aa, Artificial Sequence
  Peptide with similarity to sub-region of osteopontin
XXXXXXDXIXXXYGXXX

SEQ ID NO: 69 - protein, 15 aa, Artificial Sequence
  Peptide with similarity to sub-region of murine derived osteopontin
VDVPNGDISLAYGLR

SEQ ID NO: 70 - protein, 15 aa, Artificial Sequence
  Peptide with similarity to sub-region of murine derived osteopontin
DVPNGDISLAYGLRS

SEQ ID NO: 71 - protein, 14 aa, Artificial Sequence
  Peptide with similarity to sub-region of murine derived osteopontin
VDVPNGDISLAYGL

SEQ ID NO: 72 - protein, 14 aa, Artificial Sequence
  Peptide with similarity to sub-region of murine derived osteopontin
DVPNGDISLAYGLR

SEQ ID NO: 73 - protein, 14 aa, Artificial Sequence
  Peptide with similarity to sub-region of murine derived osteopontin
VPNGDISLAYGLRS

SEQ ID NO: 74 - protein, 13 aa, Artificial Sequence
  Peptide with similarity to sub-region of murine derived osteopontin
VDVPNGDISLAYG

SEQ ID NO: 75 - protein, 13 aa, Artificial Sequence
  Peptide with similarity to sub-region of murine derived osteopontin
DVPNGDISLAYGL

SEQ ID NO: 76 - protein, 13 aa, Artificial Sequence
  Peptide with similarity to sub-region of murine derived osteopontin
VPNGDISLAYGLR

SEQ ID NO: 77 - protein, 13 aa, Artificial Sequence
  Peptide with similarity to sub-region of murine derived osteopontin
PNGDISLAYGLRS

SEQ ID NO: 78 - protein, 12 aa, Artificial Sequence
  Peptide with similarity to sub-region of murine derived osteopontin
VDVPNGDISLAY

SEQ ID NO: 79 - protein, 12 aa, Artificial Sequence
  Peptide with similarity to sub-region of murine derived osteopontin
DVPNGDISLAYG

SEQ ID NO: 80 - protein, 12 aa, Artificial Sequence
  Peptide with similarity to sub-region of murine derived osteopontin
VPNGDISLAYGL

SEQ ID NO: 81 - protein, 12 aa, Artificial Sequence
  Peptide with similarity to sub-region of murine derived osteopontin
PNGDISLAYGLR

SEQ ID NO: 82 - protein, 12 aa, Artificial Sequence
  Peptide with similarity to sub-region of murine derived osteopontin
NGDISLAYGLRS

SEQ ID NO: 83 - protein, 11 aa, Artificial Sequence
  Peptide with similarity to sub-region of murine derived osteopontin
VDVPNGDISLA

SEQ ID NO: 84 - protein, 11 aa, Artificial Sequence
  Peptide with similarity to sub-region of murine derived osteopontin
DVPNGDISLAY

SEQ ID NO: 85 - protein, 11 aa, Artificial Sequence
  Peptide with similarity to sub-region of murine derived osteopontin
VPNGDISLAYG

SEQ ID NO: 86 - protein, 11 aa, Artificial Sequence
  Peptide with similarity to sub-region of murine derived osteopontin
PNGDISLAYGL

SEQ ID NO: 87 - protein, 11 aa, Artificial Sequence
  Peptide with similarity to sub-region of murine derived osteopontin
NGDISLAYGLR

SEQ ID NO: 88 - protein, 11 aa, Artificial Sequence
  Peptide with similarity to sub-region of murine derived osteopontin
GDISLAYGLRS

SEQ ID NO: 89 - protein, 10 aa, Artificial Sequence
  Peptide with similarity to sub-region of murine derived osteopontin
VDVPNGDISL

SEQ ID NO: 90 - protein, 10 aa, Artificial Sequence
  Peptide with similarity to sub-region of murine derived osteopontin
DVPNGDISLA

SEQ ID NO: 91 - protein, 10 aa, Artificial Sequence
  Peptide with similarity to sub-region of murine derived osteopontin
VPNGDISLAY

SEQ ID NO: 92 - protein, 10 aa, Artificial Sequence
  Peptide with similarity to sub-region of murine derived osteopontin
PNGDISLAYG

SEQ ID NO: 93 - protein, 10 aa, Artificial Sequence
  Peptide with similarity to sub-region of murine derived osteopontin
NGDISLAYGL

SEQ ID NO: 94 - protein, 10 aa, Artificial Sequence
  Peptide with similarity to sub-region of murine derived osteopontin
GDISLAYGLR

SEQ ID NO: 95 - protein, 10 aa, Artificial Sequence
  Peptide with similarity to sub-region of murine derived osteopontin
DISLAYGLRS

SEQ ID NO: 96 - protein, 9 aa, Artificial Sequence
  Peptide with similarity to sub-region of murine derived osteopontin
VDVPNGDIS

SEQ ID NO: 97 - protein, 9 aa, Artificial Sequence
  Peptide with similarity to sub-region of murine derived osteopontin
DVPNGDISL

SEQ ID NO: 98 - protein, 9 aa, Artificial Sequence
  Peptide with similarity to sub-region of murine derived osteopontin
VPNGDISLA

SEQ ID NO: 99 - protein, 9 aa, Artificial Sequence
  Peptide with similarity to sub-region of murine derived osteopontin
PNGDISLAY

SEQ ID NO: 100 - protein, 9 aa, Artificial Sequence
  Peptide with similarity to sub-region of murine derived osteopontin
NGDISLAYG

SEQ ID NO: 101 - protein, 9 aa, Artificial Sequence
  Peptide with similarity to sub-region of murine derived osteopontin
GDISLAYGL

SEQ ID NO: 102 - protein, 9 aa, Artificial Sequence
  Peptide with similarity to sub-region of murine derived osteopontin
DISLAYGLR

SEQ ID NO: 103 - protein, 9 aa, Artificial Sequence
  Peptide with similarity to sub-region of murine derived osteopontin
ISLAYGLRS

SEQ ID NO: 104 - protein, 8 aa, Artificial Sequence
  Peptide with similarity to sub-region of murine derived osteopontin
VDVPNGDI

SEQ ID NO: 105 - protein, 8 aa, Artificial Sequence
  Peptide with similarity to sub-region of murine derived osteopontin
DVPNGDIS

SEQ ID NO: 106 - protein, 8 aa, Artificial Sequence
  Peptide with similarity to sub-region of murine derived osteopontin
VPNGDISL

SEQ ID NO: 107 - protein, 8 aa, Artificial Sequence
  Peptide with similarity to sub-region of murine derived osteopontin
PNGDISLA

SEQ ID NO: 108 - protein, 8 aa, Artificial Sequence
  Peptide with similarity to sub-region of murine derived osteopontin
NGDISLAY

SEQ ID NO: 109 - protein, 8 aa, Artificial Sequence
  Peptide with similarity to sub-region of murine derived osteopontin
GDISLAYG

SEQ ID NO: 110 - protein, 8 aa, Artificial Sequence
  Peptide with similarity to sub-region of murine derived osteopontin
DISLAYGL

SEQ ID NO: 111 - protein, 8 aa, Artificial Sequence
  Peptide with similarity to sub-region of murine derived osteopontin
ISLAYGLR

SEQ ID NO: 112 - protein, 7 aa, Artificial Sequence
  Peptide with similarity to sub-region of murine derived osteopontin
VDVPNGD

SEQ ID NO: 113 - protein, 7 aa, Artificial Sequence
  Peptide with similarity to sub-region of murine derived osteopontin
DVPNGDI

SEQ ID NO: 114 - protein, 7 aa, Artificial Sequence
  Peptide with similarity to sub-region of murine derived osteopontin
VPNGDIS

SEQ ID NO: 115 - protein, 7 aa, Artificial Sequence
  Peptide with similarity to sub-region of murine derived osteopontin
PNGDISL

SEQ ID NO: 116 - protein, 7 aa, Artificial Sequence
  Peptide with similarity to sub-region of murine derived osteopontin
NGDISLA

SEQ ID NO: 117 - protein, 7 aa, Artificial Sequence
  Peptide with similarity to sub-region of murine derived osteopontin
GDISLAY

SEQ ID NO: 118 - protein, 7 aa, Artificial Sequence
  Peptide with similarity to sub-region of murine derived osteopontin
DISLAYG

SEQ ID NO: 119 - protein, 7 aa, Artificial Sequence
  Peptide with similarity to sub-region of murine derived osteopontin
ISLAYGL

SEQ ID NO: 120 - protein, 6 aa, Artificial Sequence
  Peptide with similarity to sub-region of murine derived osteopontin
DVPNGD

SEQ ID NO: 121 - protein, 6 aa, Artificial Sequence
  Peptide with similarity to sub-region of murine derived osteopontin
VPNGDI

SEQ ID NO: 122 - protein, 6 aa, Artificial Sequence
  Peptide with similarity to sub-region of murine derived osteopontin
PNGDIS

SEQ ID NO: 123 - protein, 6 aa, Artificial Sequence
  Peptide with similarity to sub-region of murine derived osteopontin
NGDISL

SEQ ID NO: 124 - protein, 6 aa, Artificial Sequence
  Peptide with similarity to sub-region of murine derived osteopontin
GDISLA

SEQ ID NO: 125 - protein, 6 aa, Artificial Sequence
  Peptide with similarity to sub-region of murine derived osteopontin
DISLAY

SEQ ID NO: 126 - protein, 6 aa, Artificial Sequence
  Peptide with similarity to sub-region of murine derived osteopontin
ISLAYG

SEQ ID NO: 127 - protein, 5 aa, Artificial Sequence
  Peptide with similarity to sub-region of murine derived osteopontin
VPNGD

SEQ ID NO: 128 - protein, 5 aa, Artificial Sequence
  Peptide with similarity to sub-region of murine derived osteopontin
PNGDI

SEQ ID NO: 129 - protein, 5 aa, Artificial Sequence
  Peptide with similarity to sub-region of murine derived osteopontin
NGDIS

SEQ ID NO: 130 - protein, 5 aa, Artificial Sequence
  Peptide with similarity to sub-region of murine derived osteopontin
GDISL

SEQ ID NO: 131 - protein, 5 aa, Artificial Sequence
  Peptide with similarity to sub-region of murine derived osteopontin
DISLA

SEQ ID NO: 132 - protein, 5 aa, Artificial Sequence
  Peptide with similarity to sub-region of murine derived osteopontin
ISLAY

SEQ ID NO: 133 - protein, 5 aa, Artificial Sequence
  Peptide with similarity to sub-region of murine derived osteopontin
SLAYG

SEQ ID NO: 134 - protein, 294 aa, Murinae
  Osteopontin
MRLAVICFCLFGIASSLPVKVTDSGSSEEKLYSLHPDPIATWLVPDPSQKQNLLAPQNAVSSEEKDDFKQETLPSNSNESHDHMDDDDDDDDDDGDHAESEDSVDSDESDESHHSDESDETVTASTQADTFTPIVPTVDVPNGRGDSLAYGLRSKSRSFQVSDEQYPDATDEDLTSHMKSGESKESLDVIPVAQLLSMPSDQDNNGKGSHESSQLDEPSLETHRLEHSKESQESADQSDVIDSQASSKASLEHQSHKFHSHKDKLVLDPKSKEDDRYLKFRISHELESSSSEVN

SEQ ID NO: 135 - protein, 16 aa, Artificial Sequence
  Peptide with similarity to sub-region of murine derived osteopontin
VDVPNGRGDSLAYGLR

SEQ ID NO: 136 - protein, 7 aa, Murinae
  Peptide derived from osteopontin
SLAYGLR

SEQ ID NO: 137 - protein, 11 aa, Murinae
  Peptide derived from osteopontin
RGDSLAYGLRS

(19) United States
(12) Patent Application Publication
(10) Pub. No.: US 2017/0143605 A1
(43) Pub. Date: May 25, 2017

(54) Compositions Comprising Osteopontin Derivatives for the Inhibition of Hair Growth

(71) Applicant: FOLLICUM AB, Lund (SE)

(72) Inventors: Jan Alenfall, Lund (SE); Pontus Duner, Lund (SE); Anna Hultgardh Nilsson, Lund (SE); Ralf Paus, Munster (DE)

(21) Appl. No.: 15/300,056

(22) PCT Filed: Apr. 17, 2015
(86) PCT No.: PCT/GB2015/051165

§ 371 Date: Sep. 28, 2016

(30) Foreign Application Priority Data
Apr. 17, 2014 (GB) 1406989.2

(51) Int. Cl. A61K 8/64; A61K 38/19; A61K 9/00; A61Q 7/02

(52) U.S. Cl. CPC A61K 8/64; A61Q 7/02; A61K 38/19; A61K 9/0014; A61K 2800/74

(57) ABSTRACT

The present invention provides a composition for inhibiting hair production in a mammal comprising an active polypeptide component and a pharmaceutically acceptable and/or cosmetically acceptable excipient, carrier or diluent. The invention further provides methods of inhibiting hair production in a mammal, e.g. human.

### FIELD OF INVENTION

The present invention relates to treatments for inhibiting hair production in mammals (including humans). In particular, there are provided polypeptide compositions for medical and cosmetic use in treating and preventing unwanted or excessive hair growth.

### BACKGROUND

Unwanted hair growth is a common problem, particularly in women, and is most often a result of ethnic background or heredity. In a small percentage of women, it may be caused by androgen overproduction, increased sensitivity to circulating androgens, or other metabolic and endocrine disorders. Approximately 22% of women are affected by the presence of unwanted hair growth on the moustache and chin area, and this can be a source of distress, leading to anxiety, depression and a reduced quality of life.

The underlying causes of unwanted hair growth may vary. Most are ethnic or hereditary; however, one must rule out any signs of androgen excess, e.g. an increase in body hair, irregular menstrual cycles, acne, alopecia, and seborrhoea.

Polycystic Ovary Syndrome (PCOS) is the most common cause of androgen excess, and 70%-80% of patients with androgen excess demonstrate hirsutism, though this sign may be less prevalent among women of Asian extraction. There is a strong familial predilection for hirsutism, primarily because the underlying endocrine disorders in this population and the factors regulating the development of hair growth have a strong genetic component.

Patients should be adequately advised of the available treatment modalities for hair removal. No single method of hair removal is appropriate for all body locations or patients, and the one adopted will depend on the character, area and amount of hair growth, as well as on the patient's age, underlying disease or condition and their personal preference as well as possible side effects of the treatment of choice.

Of course, it is also recognised that healthy individual may wish to inhibit hair growth on selected areas of their body for purely cosmetic reasons.

Treatment options for removing excess hair are limited and can vary in effectiveness, the degree of discomfort, and cost. Current methods for removing this unwanted hair include such over-the-counter methods as plucking, waxing (including the sugar forms), depilatories, shaving, and home electrolysis. Hair removal methods that could take place in a doctor's office include laser removal and intense pulsed light (IPL). An additional modality is a topical cream that inhibits hair growth: eflornithine 13.9% cream (Vaniqa®, Shire Pharmaceuticals).

These methods are temporary with the time of regrowth ranging from a few days to a few months. For hirsutism associated with PCOS, treatments include oral contraceptives and/or anti-androgens, such as spironolactone, cyproterone acetate, flutamide and finasteride.

Accordingly, there is a need for new treatments for inhibiting hair growth, suitable for use in both medical and cosmetic applications.

### SUMMARY OF INVENTION

A first aspect of the invention provides a composition for use in inhibiting hair production in a mammal comprising:
(a) a polypeptide comprising or consisting of an amino acid sequence of SEQ ID NO:1

[“FOL-005”]

SEQ ID NO: 1

VDTYDGDISVVYGLR

or a fragment or variant thereof which retains an inhibitory activity on mammalian hair production; and
(b) a pharmaceutically acceptable and/or cosmetically acceptable excipient, carrier, adjuvant or diluent.
   wherein the polypeptide is between 5 and 50 amino acids in length

The polypeptides present in the compositions of the invention are capable of inhibiting hair production in mammals.

By “inhibiting hair production” we mean that the composition is capable of reducing, attenuating, preventing or eliminating one or more of the following parameters of hair production in a mammal to which it is administered:
(a) the growth of individual hairs from existing hair follicles; and/or
(b) the thickness of individual hairs from existing hair follicles; and/or
(c) the number and/or density of existing hair follicles; and/or
(d) the production of new hair follicles; and/or
(e) the duration of the anagen phase of hair follicles; and/or
(f) the pigmentation of existing hair follicles.

It will be appreciated that such inhibition may be in whole or in part, relative to hair production in a mammal in the absence of a composition of the invention. For example, the composition may slow but not completely arrest hair growth from existing hair follicles.

Advantageously, the composition is capable of inhibiting the production of hair in vivo.

In one embodiment, the composition is capable of inhibiting the growth of human hair.

In a further embodiment, the composition is capable of inhibiting the production of hair by healthy skin.

In a further embodiment, the composition is capable of inhibiting hair production on the scalp.

As detailed above, the inhibition of hair production by the compositions of the invention may be mediated by an inhibitory effect on existing hair follicles and/or by inhibiting the formation of new hair follicles.

Thus, in one embodiment, the polypeptide is capable of inhibiting existing hair follicles by inducing:
(a) a decrease in the length of the anagen phase (e.g. by inducing an anagen to catagen phase change) in hair follicles; and/or
(b) an increase in the length of the catagen phase in hair follicles; and/or
(c) an increase in the length of the telogen phase in hair follicles.

For example, the polypeptide may be capable of inhibiting existing hair follicles by inducing a shift from the anagen phase to the catagen and/or telogen phases in existing hair follicles, thus ending active hair growth by the follicles).

The polypeptide component of the compositions of the invention shares amino acid sequence similarity with a sub-region of naturally-occurring osteopontin proteins. Thus, in some embodiments at least, the active polypeptide component may be regarded as an active fragment of a naturally-occurring osteopontin protein or a variant of such as a fragment (i.e. the polypeptide comprises an amino acid sequence corresponding to that of a modified, for example mutated, version of a fragment of a naturally-occurring osteopontin protein).

Osteopontin, also known as bone sialoprotein I (BSP-1 or BNSP), early T-lymphocyte activation (ETA-1), secreted phosphoprotein 1 (SPP1), 2ar and Rickettsia resistance (Ric), is a gene product which is conserved in several mammalian species.

The gene has seven exons, spans 5 kilobases in length and in humans it is located on the long arm of chromosome 4 region 13 (4q13). The protein is composed of ˜300 amino acids residues and is rich in acidic residues: 30-36% are either aspartic or glutamic acid. Osteopontin has about 30 attached carbohydrate residues, including 10 sialic acid residues, which are attached to the protein during post-translational modification in the Golgi apparatus.

Osteopontin was first discovered as a novel sialoprotein in bone anchoring osteoclasts onto the mineralized bone matrix (Franzen & Heinegard, 1985, Biochem. J. 232(3)715-24). The name osteopontin comes from the presence of the protein in bone (osteo-) and its ability to form a bridge (-pons) between bone cells and the mineral phase. Sequence analysis and subsequent structural studies showed osteopontin to be a 32 kDa glycoprotein composed of a highly acidic region of some ten aspartic acid residues thought to mediate the mineral binding properties of osteopontin. Furthermore, in the mid-portion of the osteopontin molecule there is also a cell attachment domain mediated through an R-G-D sequence (Oldberg et al., 1986, Proc. Natl. Acad. Sci. USA 83(23):8819-23, the disclosures of which are incorporated herein by reference).

Osteopontin is constitutively expressed in osteoblasts and in several epithelial cell types, resulting in osteopontin being secreted into many body fluids. Bone is the only tissue type where osteopontin is deposited and from where it can be recovered in large amounts. The expression of osteopontin can also be induced in vascular smooth muscle cells, in different cancer cell types and among inflammatory cells (specifically macrophages and T lymphocytes). Several important cellular functions have been attributed to osteopontin such as adhesion, proliferation, migration, anti-apoptosis and chemo attraction. Some of these functions are believed to be mediated via the RGD cell-adhesion domain which interacts with different integrins, mainly with ανβ3 but also ανβ1, and ανβ5 (for review see Scatena et al., 2007, Arterio. Thromb. Vasc. Biol. 27:2302-2309).

In recent years, osteopontin has emerged as a potent cytokine capable of modulating several cell types involved in inflammation and immune responses. The broad range of functions being attributed to osteopontin has been puzzling and cannot all be explained by the single cell-binding RGD sequence. The explanation came when an eleven amino acid peptide in osteopontin R¹⁴⁵-G-D-S-L-A-Y-G-L-R-S¹⁵⁵ (SEQ ID NO:135) (corresponding to amino acids 144 to 154 of UniProt code P10923) was identified and later functionally mapped. In addition to the known R¹⁴⁵-G-D¹⁴⁷ site mediating binding to the ανβ3 integrin, two additional essential regions in the osteopontin molecule were discovered, namely a highly specific thrombin cleavage site, i.e. R¹⁵⁴-S¹⁵⁵, and a cryptic integrin binding site, i.e. S¹⁴⁸-L-A-Y-G-L-R¹⁵⁴ (SEQ ID NO:136), which binds to an α9β1 integrin (see Scatena et al., supra). An additional binding site for α4β1 has also been identified (see Scatena et al., supra).

Naturally-occurring osteopontin proteins are typically around 300 amino acids in length. However, the polypeptide components of the compositions of the invention are considerably shorter in length, being from 5 to 50 amino acids only.

Thus, the polypeptide is fewer than 50 amino acids in length, for example fewer than 35, 30, 28, 26, 24, 22, 20, 19, 18, 17, 16, 15, 14, 13, 12, 11, or 10 amino acids in length.

In one embodiment, the polypeptide is between 5 and 30 amino acids in length, for example between 5 and 20, between 5 and 20, between 8 and 20, between 8 and 16, or between 10 and 15 amino acids in length.

The term ‘amino acid’ as used herein includes the standard twenty genetically-encoded amino acids and their corresponding stereoisomers in the ‘D’ form (as compared to the natural ‘L’ form), omega-amino acids and other naturally-occurring amino acids, unconventional amino acids (e.g., α,α-disubstituted amino acids, N-alkyl amino acids, etc.) and chemically derivatised amino acids (see below).

When an amino acid is being specifically enumerated, such as ‘alanine’ or ‘Ala’ or ‘A’, the term refers to both L-alanine and D-alanine unless explicitly stated otherwise. Other unconventional amino acids may also be suitable components for polypeptides of the present invention, as long as the desired functional property is retained by the polypeptide. For the polypeptides shown, each encoded amino acid residue, where appropriate, is represented by a single letter designation, corresponding to the trivial name of the conventional amino acid.

In accordance with convention, the amino acid sequences disclosed herein are provided in the N-terminus to C-terminus direction.

Typically, the polypeptides used in the compositions of the invention comprise or consist of L-amino acids.

In one preferred embodiment, the polypeptide component of the compositions of the invention consists of an amino acid sequence of SEQ ID NO:1.

In an alternative embodiment, the polypeptide component of the compositions of the invention consists of an amino acid sequence of SEQ ID NO:2.

SEQ ID NO: 2

VDTYDGDISVVYGLS

In a further embodiment, the polypeptide may comprise or consist of a fragment of the amino acid sequence of SEQ ID NO: 1 which retains (at least in part) an inhibitory activity on hair production.

By “fragment” we include at least 5 contiguous amino acids of the amino acid sequence of SEQ ID NO: 1, for example at least 6, 7, 8, 9, 10, 11, 12, 13, or 14 contiguous amino acids of SEQ ID NO: 1. Thus, the fragment may be 14 or fewer amino acids in length, for example 13, 12, 11, 10, 9, 8, 7, 6 or 5 amino acids in length

In exemplary embodiments, the fragment comprises or consists of an amino acid sequence according to any one SEQ ID NOs: 3 to 65
(a) 14-amino acid peptides:

SEQ ID NO: 3

VDTYDGDISVVYGL

SEQ ID NO: 4

DTYDGDISVVYGLR

SEQ ID NO: 5

TYDGDISVVYGLRS

(b) 13-amino acid peptides:

SEQ ID NO: 6

VDTYDGDISVVYG

SEQ ID NO: 7

DTYDGDISVVYGL

SEQ ID NO: 8

TYDGDISVVYGLR

SEQ ID NO: 9

YDGDISVVYGLRS

(c) 12-amino acid peptides:

SEQ ID NO: 10

VDTYDGDISVVY

SEQ ID NO: 11

DTYDGDISVVYG

SEQ ID NO: 12

TYDGDISVVYGL

SEQ ID NO: 13

YDGDISVVYGLR

SEQ ID NO: 14

DGDISVVYGLRS

(d) 11-amino acid peptides:

SEQ ID NO: 15

VDTYDGDISVV

SEQ ID NO: 16

DTYDGDISVVY

SEQ ID NO: 17

TYDGDISVVYG

SEQ ID NO: 18

YDGDISVVYGL

SEQ ID NO: 19

DGDISVVYGLR

SEQ ID NO: 20

GDISVVYGLRS

(e) 10-amino acid peptides:

SEQ ID NO: 21

VDTYDGDISV

SEQ ID NO: 22

DTYDGDISVV

SEQ ID NO: 23

TYDGDISVVY

SEQ ID NO: 24

YDGDISVVYG

SEQ ID NO: 25

DGDISVVYGL

SEQ ID NO: 26

GDISVVYGLR

SEQ ID NO: 27

DISVVYGLRS

(f) 9-amino acid peptides:

SEQ ID NO: 28

VDTYDGDIS

SEQ ID NO: 29

DTYDGDISV

SEQ ID NO: 30

TYDGDISVV

SEQ ID NO: 31

YDGDISVVY

SEQ ID NO: 32

DGDISVVYG

SEQ ID NO: 33

GDISVVYGL

SEQ ID NO: 34

DISVVYGLR

SEQ ID NO: 35

ISVVYGLRS

(g) 8-amino acid peptides:

SEQ ID NO: 36

VDTYDGDI

SEQ ID NO: 37

DTYDGDIS

SEQ ID NO: 38

TYDGDISV

SEQ ID NO: 39

YDGDISVV

SEQ ID NO: 40

DGDISVVY

SEQ ID NO: 41

GDISVVYG

SEQ ID NO: 42

DISVVYGL

SEQ ID NO: 43

ISVVYGLR

(h) 7-amino acid peptides:

SEQ ID NO: 44

VDTYDGD

SEQ ID NO: 45

DTYDGDI

SEQ ID NO: 46

TYDGDIS

SEQ ID NO: 47

YDGDISV

SEQ ID NO: 48

DGDISVV

SEQ ID NO: 49

GDISVVY

SEQ ID NO: 50

DISVVYG

SEQ ID NO: 51

ISVVYGL

(i) 6-amino acid peptides:

SEQ ID NO: 52

DTYDGD

SEQ ID NO: 53

TYDGDI

SEQ ID NO: 54

YDGDIS

SEQ ID NO: 55

DGDISV

SEQ ID NO: 56

GDISVV

SEQ ID NO: 57

DISVVY

SEQ ID NO: 58

ISVVYG

(j) 5-amino acid peptides:

SEQ ID NO: 59

TYDGD

SEQ ID NO: 60

YDGDI

SEQ ID NO: 61

DGDIS

SEQ ID NO: 62

GDISV

SEQ ID NO: 63

DISVV

SEQ ID NO: 64

ISVVY

SEQ ID NO: 65.

SVVYG

For example, the fragment may comprise or consist of an amino acid sequence of SEQ ID NO: 26.

Alternatively, the compositions of the invention may comprise a polypeptide which comprises or consists of a variant of the amino acid sequence of SEQ ID NO: 1, or of a fragment thereof, which retains (at least in part) the inhibitory activity on hair production.

By “variant” we mean that the polypeptide does not share 100% amino acid sequence identity with SEQ ID NO: 1, i.e. one or more amino acids of SEQ ID NO: 1 must be mutated. For example, the polypeptide may comprise or consist of an amino acid sequence with at least 50% identity to the amino acid sequence of SEQ ID NO: 1, more preferably at least 60%, 70% or 80% or 85% or 90% identity to said sequence, and most preferably at least 95%, 96%, 97%, 98% or 99% identity to said amino acid sequence.

Percent identity can be determined by methods well known in the art, for example using the LALIGN program (Huang and Miller, Adv. Appl. Math. (1991) 12:337-357) at the Expasy facility site
(http://www.ch.embnet.orq/software/LALIGN_form.html)
   using as parameters the global alignment option, scoring matrix BLOSUM62, opening gap penalty −14, extending gap penalty −4.

Alternatively, the percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

By “mutated” we mean that the amino acid at the specified position is altered compared to the amino acid in the polypeptide according to SEQ ID NO: 1. For example, an amino acid at a specified position may be deleted, substituted or may be the site of an insertion/addition of one or more amino acids. It will be appreciated by persons skilled in the art that the substitutions may be conservative or non-conservative.

Alternatively, or in addition, the amino acid at a specified position may be chemically modified (derivatised); see below.

In one embodiment, the variant polypeptide comprises or consists of an amino acid sequence of SEQ ID NO: 1, or a fragment thereof, in which one or more amino acids is conservatively substituted. By “conservatively substituted” we mean a substitution of one amino acid with another with similar properties (size, hydrophobicity, etc), such that the function of the polypeptide is not significantly altered. Thus, by “conservative substitutions” is intended combinations such as Gly, Ala; Val, Ile, Leu; Asp, Glu; Asn, Gln; Ser, Thr; Lys, Arg; and Phe, Tyr.

It will be appreciated by persons skilled in the art that the variant polypeptide may comprise one or more additional amino acids, inserted at the N- and/or C-terminus and/or internally within the amino acid sequence of SEQ ID NO:1. For example, the polypeptide may comprise or consist of at least 2, 3, 4, 5, 6, 7, 8, 9, 10, 15 or 20 additional amino acids at the N- and/or C-terminus and/or internally. The variant polypeptide may be naturally occurring or non-naturally occurring.

In one preferred embodiment, the additional amino acids are the amino acids from the corresponding positions of the wildtype human osteopontin, i.e. GenBank: AAA59974.1 [SEQ ID NO:66] (wherein the region of highest sequence similarity to SEQ ID NO:1 is underlined in italics):

SEQ ID NO: 66

MRIAVICFCLLGITCAIPVKQADSGSSEEKQLYNKYPDAVATWLNPDPSQ
KQNLLAPQTLPSKSNESHDHMDDMDDEDDDDHVDSQDSIDSNDSDDVDDT
DDSHQSDESHHSDESDELVTDFPTDLPATEVFTPVVPTVDTYDGRGDSVV
YGLRSKSKKFRRPDIQYPDATDEDITSHMESEELNGAYKAIPVAQDLNAP
SDWDSRGKDSYETSQLDDQSAETHSHKQSRLYKRKANDESNEHSDVIDSQ
ELSKVSREFHSHEFHSHEDMLVVDPKSKEEDKHLKFRISHELDSASSEVN

By “corresponding positions” of the wildtype human osteopontin we mean that the additional amino acids are the same as those present in the equivalent position in the above wildtype human osteopontin (if one imagines that the amino acid sequence of SEQ ID NO:1 replaces the sequence underlined in italics in SEQ ID NO:66). For example, the polypeptide may comprise the three amino acids VPT- at the amino terminus of SEQ ID NO:1 and/or the five amino acids -SKSKK at the carboxy terminus of SEQ ID NO:1.

In a further embodiment, the variant polypeptide sequence may comprise or consist of an amino acid sequence of SEQ ID NO: 67, or of a fragment or variant thereof

SEQ ID NO: 67

VDTYDGRGDSVVYGLR

Suitable fragments may consist of 15 or fewer contiguous amino acids of SEQ ID NO: 67, for example 14, 13, 12, 11, 10, 9, 8, 7, 6 or 5 amino acids continuous amino acids of SEQ ID NO: 67.

Likewise, suitable variants (as defined above) may comprise or consist of an amino acid sequence with at least 50% identity to the amino acid sequence of SEQ ID NO:67, more preferably at least 60%, 70% or 80% or 85% or 90% identity to said sequence, and most preferably at least 95%, 96%, 97%, 98% or 99% identity to said amino acid sequence.

In one embodiment, the variant comprises or consists of an amino acid sequence of SEQ ID NO:67, or a fragment thereof, in which the RGD sequence is inactivated.

The active tri-peptide sequence “arginine-glycine-aspartic acid” normally found in naturally-occurring osteopontin proteins may be inactivated by a number of different strategies. In one embodiment, the RGD domain is mutated at one or more amino acids in the polypeptide, such that it contains one or more deletions, substitutions and/or additions, or combinations thereof, relative to a naturally-occurring osteopontin protein. For example, the RGD domain may be deleted, at least in part, such that the arginine and/or glycine and/or aspartic acid residue is absent.

Alternatively, or in addition, the RGD domain may be substituted at one or more amino acids. For example, the arginine and/or glycine and/or aspartic acid residue may be substituted with another amino acid. Such substitutions may be conservative or non-conservative.

Likewise, the RGD domain may be inactivated by a combination of substitutions and deletions, including:
(a) substitution of the arginine residue and deletion of the glycine and aspartic acid residues;
(b) substitution of the arginine and glycine residues and deletion of the aspartic acid residue (for example, the -RGD- tripeptide can be replaced with the dipeptide sequence -DI-);
(c) substitution of the arginine and aspartic acid residues and deletion of the glycine residue; or
(d) deletion of the arginine and aspartic acid residues and substitution of the glycine residue.

In one embodiment, the tripeptide -RGD- sequence is replaced by the dipeptide -DI-sequence.

Without wishing to be bound by theory, it is believed that inactivation of the RGD sequence may result in a conformational change in the osteopontin polypeptide (relative to the corresponding naturally-occurring osteopontin protein), which leads to the creation/exposure of a new site to the surrounding milieu.

The above-defined polypeptide components of the compositions of the invention are derived from the amino acid sequence of human osteopontin, and mutated variants thereof. However, it will be appreciated by persons skilled in the art that the polypeptide may alternatively be a species homologue of the amino acid sequence of SEQ ID NO:1 (for example, the homologue from mouse, cattle, pig, rabbit, rat, etc.).

The amino acid sequence of such species homologues of SEQ ID NO:1 may be represented by the following formula:

SEQ ID NO: 68

X1-X2-X3-X4-X5-X6-D-X8-I-X10-X11-X12-Y-G-X15-X16-
X17

wherein
X1 is V, E, G or K;
X2 is D, E, S or P;
X3 is any amino acid (e.g. T, V, P, A or L);
X4 is Y, P, N or A;
X5 is D, Nor E;
X6 is G or I;
X8 is G or absent;
X10 is S or E;
X11 is V or L;
X12 is V, A or T;
X15 is L or I;
X16 is R or K; and
X17 is R, K or absent.

It will be appreciated by persons skilled in the art that the variant polypeptide may comprise one or more additional amino acids, added at the N- and/or C-terminus and/or internally within the amino acid sequence of SEQ ID NO:68. For example, the polypeptide may comprise or consist of at least 2, 3, 4, 5, 6, 7, 8, 9, 10, 15 or 20 additional amino acids at the N- and/or C-terminus and/or internally. In one preferred embodiment, the additional amino acids are the amino acids from the corresponding positions of a wildtype osteopontin, i.e. from human, mouse, cattle, pig, rabbit, rat, etc.)

Thus, in one embodiment, the polypeptide is a murine homologue which comprises or consists of an amino acid sequence of SEQ ID NO:69 or 70, or of a fragment or variant thereof:

SEQ ID NO: 69

VDVPNGDISLAYGLR

SEQ ID NO: 70

DVPNGDISLAYGLRS

Suitable fragments may consist of 14 or fewer contiguous amino acids of SEQ ID NO: 69 or 70, for example 13, 12, 11, 10, 9, 8, 7, 6 or 5 amino acids continuous amino acids of SEQ ID NO: 69 or 70.

For example, the fragment may be comprise or consist of an amino acid sequence according to any one SEQ ID NOs: 71 to 133:
(a) 14-amino acid peptides:

SEQ ID NO: 71

VDVPNGDISLAYGL

SEQ ID NO: 72

DVPNGDISLAYGLR

SEQ ID NO: 73

VPNGDISLAYGLRS

(b) 13-amino acid peptides:

SEQ ID NO: 74

VDVPNGDISLAYG

SEQ ID NO: 75

DVPNGDISLAYGL

SEQ ID NO: 76

VPNGDISLAYGLR

SEQ ID NO: 77

PNGDISLAYGLRS

(c) 12-amino acid peptides:

SEQ ID NO: 78

VDVPNGDISLAY

SEQ ID NO: 79

DVPNGDISLAYG

SEQ ID NO: 80

VPNGDISLAYGL

SEQ ID NO: 81

PNGDISLAYGLR

SEQ ID NO: 82

NGDISLAYGLRS

(d) 11-amino acid peptides:

SEQ ID NO: 83

VDVPNGDISLA

SEQ ID NO: 84

DVPNGDISLAY

SEQ ID NO: 85

VPNGDISLAYG

SEQ ID NO: 86

PNGDISLAYGL

SEQ ID NO: 87

NGDISLAYGLR

SEQ ID NO: 88

GDISLAYGLRS

(e) 10-amino acid peptides:

SEQ ID NO: 89

VDVPNGDISL

SEQ ID NO: 90

DVPNGDISLA

SEQ ID NO: 91

VPNGDISLAY

SEQ ID NO: 92

PNGDISLAYG

SEQ ID NO: 93

NGDISLAYGL

SEQ ID NO: 94

GDISLAYGLR

SEQ ID NO: 95

DISLAYGLRS

(f) 9-amino acid peptides:

SEQ ID NO: 96

VDVPNGDIS

SEQ ID NO: 97

DVPNGDISL

SEQ ID NO: 98

VPNGDISLA

SEQ ID NO: 99

PNGDISLAY

SEQ ID NO: 100

NGDISLAYG

SEQ ID NO: 101

GDISLAYGL

SEQ ID NO: 102

DISLAYGLR

SEQ ID NO: 103

ISLAYGLRS

(g) 8-amino acid peptides:

SEQ ID NO: 104

VDVPNGDI

SEQ ID NO: 105

DVPNGDIS

SEQ ID NO: 106

VPNGDISL

SEQ ID NO: 107

PNGDISLA

SEQ ID NO: 108

NGDISLAY

SEQ ID NO: 109

GDISLAYG

SEQ ID NO: 110

DISLAYGL

SEQ ID NO: 111

ISLAYGLR

(h) 7-amino acid peptides:

SEQ ID NO: 112

VDVPNGD

SEQ ID NO: 113

DVPNGDI

SEQ ID NO: 114

VPNGDIS

SEQ ID NO: 115

PNGDISL

SEQ ID NO: 116

NGDISLA

SEQ ID NO: 117

GDISLAY

SEQ ID NO: 118

DISLAYG

SEQ ID NO: 119

ISLAYGL

(i) 6-amino acid peptides:

SEQ ID NO: 120

DVPNGD

SEQ ID NO: 121

VPNGDI

SEQ ID NO: 122

PNGDIS

SEQ ID NO: 123

NGDISL

SEQ ID NO: 124

GDISLA

SEQ ID NO: 125

DISLAY

SEQ ID NO: 126

ISLAYG

(j) 5-amino acid peptides:

SEQ ID NO: 127

VPNGD

SEQ ID NO: 128

PNGDI

SEQ ID NO: 129

NGDIS

SEQ ID NO: 130

GDISL

SEQ ID NO: 131

DISLA

SEQ ID NO: 132

ISLAY

SEQ ID NO: 133

SLAYG

In embodiment, the fragment comprises or consists of an amino acid sequence of SEQ ID NO:94.

Likewise, the polypeptide may comprise or consist of a variant of the amino acid sequence of SEQ ID NO: 69 or 70, or of a fragment thereof.

Suitable variants (as defined above) may comprise or consist of an amino acid sequence with at least 50% identity to the amino acid sequence of SEQ ID NO: 69 or 70, more preferably at least 60%, 70% or 80% or 85% or 90% identity to said sequence, and most preferably at least 95%, 96%, 97%, 98% or 99% identity to said amino acid sequence.

The variant may comprise or consist of an amino acid sequence of SEQ ID NO: 69 or 70, or a fragment thereof, in which one or more amino acids is conservatively substituted.

Optionally, the polypeptide may comprise or consist of one or more additional amino acids, inserted at the N- and/or C-terminus and/or internally within the amino acid sequence of SEQ ID NO:69 or 70. For example, the polypeptide may comprise or consist of at least 2, 3, 4, 5, 6, 7, 8, 9, 10, 15 or 20 additional amino acids, which may from the corresponding positions of the wildtype murine osteopontin, i.e. NCBI Reference Sequence: NP_001191162.1 [SEQ ID NO:134] (wherein the region of highest sequence similarity to SEQ ID NO:68 is underlined in italics):

SEQ ID NO: 134

MRLAVICFCLFGIASSLPVKVTDSGSSEEKLYSLHPDPIATWLVPDPSQ
KQNLLAPQNAVSSEEKDDFKQETLPSNSNESHDHMDDDDDDDDDDGDH
AESEDSVDSDESDESHHSDESDETVTASTQADTFTPIVPTVDVPNGRGD
SLAYGLRSKSRSFQVSDEQYPDATDEDLTSHMKSGESKESLDVIPVAQL
LSMPSDQDNNGKGSHESSQLDEPSLETHRLEHSKESQESADQSDVIDS
QASSKASLEHQSHKFHSHKDKLVLDPKSKEDDRYLKFRISHELESSSSE
VN

In one embodiment, the polypeptide comprises or consists of an amino acid sequence of SEQ ID NO: 135, or of a fragment or variant thereof

SEQ ID NO: 135

VDVPNGRGDSLAYGLR

Suitable fragments may consist of 15 or fewer contiguous amino acids of SEQ ID NO: 135, for example 14, 13, 12, 11, 10, 9, 8, 7, 6 or 5 amino acids continuous amino acids of SEQ ID NO: 135.

Likewise, suitable variants (as defined above) may comprise or consist of an amino acid sequence with at least 50% identity to the amino acid sequence of SEQ ID NO: 135, more preferably at least 60%, 70% or 80% or 85% or 90% identity to said sequence, and most preferably at least 95%, 96%, 97%, 98% or 99% identity to said amino acid sequence.

In one embodiment, the variant comprises or consists of an amino acid sequence of SEQ ID NO: 135, or a fragment thereof, in which the RGD sequence is inactivated.

In a further embodiment of the compositions of the invention, the polypeptide comprises or consists of tandem repeats.

Thus, the tandem repeats may comprise or consist of the amino acid sequence of any one or more of SEQ ID NOS: 1 to 65 (for example, the tandem repeats may comprise or consist of the amino acid sequence of SEQ ID NO:1 or 26).

Alternatively, the tandem repeats may comprise or consist of the amino acid sequence of any one or more of SEQ ID NOS: 69 to 133 (for example, the tandem repeats may comprise or consist of the amino acid sequence of SEQ ID NO: 69 and/or 94).

It will be appreciated by persons skilled in the art that the polypeptide component of the compositions of the invention may be modified or derivatised at one or more amino acid positions.

Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be further appreciated by persons skilled in the art that peptidomimetic compounds may also be useful, which mimic the conformation and desirable features of the polypeptides detailed above. Thus, by ‘polypeptide’ we include peptidomimetic compounds which have a hair growth inhibitory activity of the polypeptide of SEQ ID NO: 1.

For example, the polypeptides of the invention include not only molecules in which amino acid residues are joined by peptide (—CO—NH—) linkages but also molecules in which the peptide bond is reversed. Such retro-inverso peptidomimetics may be made using methods known in the art, for example such as those described in Meziere et al. (1997) J. Immunol. 159, 3230-3237, which is incorporated herein by reference. This approach involves making pseudopeptides containing changes involving the backbone, and not the orientation of side chains. Retro-inverse peptides, which contain NH—CO bonds instead of CO—NH peptide bonds, are much more resistant to proteolysis. Alternatively, the polypeptide of the invention may be a peptidomimetic compound wherein one or more of the amino acid residues are linked by a -y(CH₂NH)— bond in place of the conventional amide linkage.

In a further alternative, the peptide bond may be dispensed with altogether provided that an appropriate linker moiety which retains the spacing between the carbon atoms of the amino acid residues is used; it may be advantageous for the linker moiety to have substantially the same charge distribution and substantially the same planarity as a peptide bond.

It will be appreciated that the polypeptide may conveniently be blocked at its N- or C-terminus so as to help reduce susceptibility to exoproteolytic digestion.

A variety of uncoded or modified amino acids such as D-amino acids and N-methyl amino acids have also been used to modify mammalian polypeptides. In addition, a presumed bioactive conformation may be stabilised by a covalent modification, such as cyclisation or by incorporation of lactam or other types of bridges, for example see Veber et al., 1978, Proc. Natl. Acad. Sci. USA 75:2636 and Thursell et al., 1983, Biochem. Biophys. Res. Comm. 111:166, which are incorporated herein by reference.

A common theme among many of the synthetic strategies has been the introduction of some cyclic moiety into a peptide-based framework. The cyclic moiety restricts the conformational space of the peptide structure and this frequently results in an increased specificity of the peptide for a particular biological receptor. An added advantage of this strategy is that the introduction of a cyclic moiety into a peptide may also result in the peptide having a diminished sensitivity to cellular peptidases.

Thus, the polypeptides of the invention may comprise terminal cysteine amino acids. Such a polypeptide may exist in a heterodetic cyclic form by disulphide bond formation of the mercaptide groups in the terminal cysteine amino acids or in a homodetic form by amide peptide bond formation between the terminal amino acids. As indicated above, cyclising small peptides through disulphide or amide bonds between the N- and C-terminal region cysteines may circumvent problems of specificity and half-life sometime observed with linear peptides, by decreasing proteolysis and also increasing the rigidity of the structure, which may yield higher specificity compounds. Polypeptides cyclised by disulphide bonds have free amino and carboxy-termini which still may be susceptible to proteolytic degradation, while peptides cyclised by formation of an amide bond between the N-terminal amine and C-terminal carboxyl and hence no longer contain free amino or carboxy termini. Thus, the peptides of the present invention can be linked either by a C—N linkage or a disulphide linkage.

The present invention is not limited in any way by the method of cyclisation of peptides, but encompasses peptides whose cyclic structure may be achieved by any suitable method of synthesis. Thus, heterodetic linkages may include, but are not limited to formation via disulphide, alkylene or sulphide bridges. Methods of synthesis of cyclic homodetic peptides and cyclic heterodetic peptides, including disulphide, sulphide and alkylene bridges, are disclosed in U.S. Pat. No. 5,643,872, which is incorporated herein by reference. Other examples of cyclisation methods includes cyclization through click chemistry, epoxides, aldehyde-amine reactions, as well as and the methods disclosed in U.S. Pat. No. 6,008,058, which is incorporated herein by reference.

Such terminal modifications are useful, as is well known, to reduce susceptibility by proteinase digestion and therefore to prolong the half-life of the peptides in solutions, particularly in biological fluids where proteases may be present. Polypeptide cyclisation is also a useful modification because of the stable structures formed by cyclisation and in view of the biological activities observed for cyclic peptides.

Thus, in one embodiment the polypeptide of the first aspect of the invention is cyclic.

However, in an alternative embodiment, the polypeptide is linear.

In one preferred embodiment, however, the polypeptide comprises one or more amino acids modified or derivatised by PEGylation, amidation, esterification, acylation, acetylation and/or alkylation.

Persons skilled in the art will appreciate that the polypeptide may be glycosylated at one or more amino acids. For example, the polypeptide may retain one or more of the glycosylation sites of the corresponding (‘parent’) osteopontin protein, to which may be attached a carbohydrate group.

In a further embodiment, the polypeptide comprises or consists of a fusion, e.g. of the amino acid sequence of SEQ ID NO: 1, 26, 69 or 94, or of a fragment or variant thereof.

For example, the polypeptide may comprise a fusion of the amino acid sequence of SEQ ID NO: 1 or 26

By ‘fusion’ of a polypeptide we include an amino acid sequence corresponding to, for example, SEQ ID NO: 1 or 26 (or a fragment or variant thereof) fused to any other polypeptide. For example, the said polypeptide may be fused to a polypeptide such as glutathione-S-transferase (GST) or protein A in order to facilitate purification of said polypeptide. Examples of such fusions are well known to those skilled in the art. Similarly, the said polypeptide may be fused to an oligo-histidine tag such as His6 or to an epitope recognised by an antibody such as the well-known Myc tag epitope. Fusions to any variant or derivative of said polypeptide are also included in the scope of the invention.

The fusion may comprise a further portion which confers a desirable feature on the said polypeptide of the invention; for example, the portion may be useful in augmenting or prolonging the hair growth inhibitory effect. For example, in one embodiment the fusion comprises human serum albumin or similar protein (as disclosed in US 2009/0005312, the disclosures of which are incorporated herein by reference).

Alternatively, the fused portion may be a lipophilic molecule or polypeptide domain that is capable of promoting cellular uptake of the polypeptide, as known to those skilled in the art.

Polypeptides suitable for use in the compositions of the invention may be made by in vitro cell-based expression methods well known to persons skilled in the art (for example, see Green & Sambrook, 2012, Molecular Cloning, A Laboratory Manual, Fourth Edition, Cold Spring Harbor, N.Y., the relevant disclosures in which document are hereby incorporated by reference). The choice of expression vector and host cell to be used may depend on a number of factors. For example, if the polypeptide is to be glycosylated, a mammalian expression system will be required.

Suitable expression vectors and host cells are commercially available from many sources.

Alternatively, the polypeptides may be synthesised by known means, such as liquid phase and solid phase synthesis (for example, t-Boc solid-phase peptide synthesis and BOP-SPPS).

It will be appreciated by persons skilled in the art that the present invention also includes the use of pharmaceutically and/or cosmetically acceptable acid or base addition salts of the above described polypeptides. The acids which are used to prepare the pharmaceutically and/or cosmetically acceptable acid addition salts of the aforementioned base compounds useful in this invention are those which form non-toxic acid addition salts, i.e. salts containing pharmaceutically and/or cosmetically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others.

Pharmaceutically and/or cosmetically acceptable base addition salts may also be used to produce pharmaceutically and/or cosmetically acceptable salt forms of the polypeptides. The chemical bases that may be used as reagents to prepare pharmaceutically and/or cosmetically acceptable base salts of the present compounds that are acidic in nature are those that form non-toxic base salts with such compounds. Such non-toxic base salts include, but are not limited to those derived from such pharmaceutically and/or cosmetically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others.

It will be further appreciated that the polypeptides may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of activity loss and that use levels may have to be adjusted upward to compensate. Preferably, the lyophilised (freeze dried) polypeptide loses no more than about 20%, or no more than about 25%, or no more than about 30%, or no more than about 35%, or no more than about 40%, or no more than about 45%, or no more than about 50% of its activity (prior to lyophilisation) when rehydrated.

The polypeptides are provided in the form of a composition comprising the polypeptide and a pharmaceutically acceptable and/or cosmetically acceptable excipient, carrier or diluent, selected with regard to the intended route of administration and standard pharmaceutical or cosmetic practice (for example, see Remington: The Science and Practice of Pharmacy, 19^{th} edition, 1995, Ed. Alfonso Gennaro, Mack Publishing Company, Pennsylvania, USA, which is incorporated herein by reference).

By “pharmaceutically acceptable” is included that the formulation is sterile and pyrogen free. Suitable pharmaceutical carriers are well known in the art of pharmacy. The carrier(s) must be “acceptable” in the sense of being compatible with the compound of the invention and not deleterious to the recipients thereof. Typically, the carriers will be water or saline which will be sterile and pyrogen free; however, other acceptable carriers may be used. Thus, “pharmaceutically acceptable carrier” and “pharmaceutically acceptable excipient” includes any compound(s) used in forming a part of the formulation that is intended to act merely as a carrier, i.e., not intended to have biological activity itself. The pharmaceutically acceptable carrier or excipient is generally safe, non-toxic, and neither biologically nor otherwise undesirable. A pharmaceutically acceptable carrier or excipient as used herein includes both one and more than one such carrier or excipient.

Likewise, the term “cosmetically acceptable” is used to denote formulations suitable for use as cosmetic products. Suitable cosmetic carriers are well known in the art, such as those commonly used in shampoos, lotions, creams and other such products.

The excipient may be one or more of carbohydrates, polymers, lipids and minerals. Examples of carbohydrates include lactose, sucrose, mannitol, and cyclodextrines, which are added to the composition, e.g. for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The term “diluent” is intended to mean an aqueous or non-aqueous solution with the purpose of diluting the peptide in the pharmaceutical preparation. The diluent may be one or more of saline, water, polyethylene glycol, propylene glycol, ethanol or oils (such as safflower oil, corn oil, peanut oil, cottonseed oil or sesame oil).

The diluent may also function as a buffer. The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

Optionally, the composition may comprise an adjuvant. The term “adjuvant” is intended to mean any compound added to the formulation to increase the biological effect of the peptide. The adjuvant may be one or more of zinc, copper or silver salts with different anions, for example, but not limited to fluoride, chloride, bromide, iodide, tiocyanate, sulfite, hydroxide, phosphate, carbonate, lactate, glycolate, citrate, borate, tartrate, and acetates of different acyl composition.

The pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(carprolactone) (PCL), and polyanhydrides have been widely used as biodegradable polymers in the production of microspheres. Preparations of such microspheres can be found in U.S. Pat. No. 5,851,451 and in EP0213303.

The pharmaceutical compositions of the invention may also be in the form of polymer gels, where polymers such as starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polysulphonate,

The polypeptides may be formulated at various concentrations, depending on the efficacy/toxicity of the particular polypeptide being used. Preferably, the composition comprises the polypeptide at a concentration of between 1 nM and 1 M, for example between 0.1 μM and 1 mM, 1 μM and 100 μM, between 5 μM and 50 μM, between 10 μM and 50 μM, between 20 μM and 40 μM and optionally about 30 μM. For ex vivo and in vitro applications, compositions may comprise a lower concentration of a polypeptide, for example between 0.0025 μM and 1 μM, between 10 nM and 300 nM or between 15 nM and 150 nM.

It will be appreciated by persons skilled in the art that the compositions of the invention may be administered by a variety of routes, for example topical, transdermal, parenteral or oral administration.

Advantageously, the compositions of the invention are suitable for topical administration or intracutaneous administration.

Thus, the compositions of the invention may be administered topically to the skin (e.g. face, legs, etc.). For example, the composition may be provided in the form of an ointment containing the active polypeptide suspended or dissolved in, for example, a mixture with one or more of the following: mineral oil, liquid petrolatum, white petrolatum, propylene glycol, polyoxyethylene polyoxypropylene compound, emulsifying wax and water. Alternatively, the polypeptide can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Optionally, the composition for topical administration may comprise a penetration enhancer (for example, as described in Osborne & Henke, 1997, Pharmaceutical Technology, November: 58-82 and Pathan & Setty, 2009, Tropical Journal of Pharmaceutical Research 8 (2): 173-179, the disclosures of which are incorporated herein by reference).

Alternatively, the compositions of the invention may be administered parenterally, for example intracutaneously. Such compositions are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

It may be beneficial to use a sustained-release system, such as microsphere formulations, for delivering the polypeptides.

It may also be useful to use micro-needles and other devices for delivering the polypeptides. Examples of suitable micro-needles include the Hollow Microneedle Technology (from 3M) and the Micro-Trans Microneedle Array Patch (from Valeris). Other formats of device for delivering the polypeptides include transdermal patches and transdermal gels. Examples of suitable transdermal patches include those adhesives available from Dow Corning. Examples of transdermal gels include those used for the transdermal administration of hormones. See also Prausnitz & Langer, 2008, Nature Biotechnology 26:1261-1268; Jain et al, 2014, Crit. Rev. Ther. Drug Carrier Syst. 31(3):219-72, and Wu et al, 2012, Curr Pharm Biotechnol. 13(7):1292-8 (the disclosures of which are incorporated herein by reference).

Alternatively, compositions can be administered by a surgically implanted device that releases the active polypeptide directly to the required site (i.e. the epidermis).

The compositions of the invention may also be delivered by transdermal methodologies.

For example, electroporation therapy (EPT) and/or iontophoresis systems can be employed for the administration of proteins and polypeptides. In such methods, a device is used to deliver a pulsed electric field to cells, resulting in the increased permeability of the cell membranes to the drug and significant enhancement of intracellular drug delivery.

An alternative transdermal method, electroincorporation, utilises small particles of up to 30 microns in diameter on the surface of the skin experience electrical pulses identical or similar to those used in electroporation. The particles are driven through the stratum corneum and into deeper layers of the skin. The particles can be loaded or coated with drugs or genes or can simply act as “bullets” that generate pores in the skin through which the drugs can enter.

Additional transdermal methodologies have also been developed by PowderJect Pharmaceuticals (owned by Novartis AG).

Suitable methods for administration of the polypeptides and compositions of the invention are well known in the art, for example, see Therapeutic Protein and Peptide Formulation and Delivery, Zahra Shahrokh et al. (Eds), 1997, American Chemical Society, ISBN13: 9780841235281.

The compositions of the invention are for use in inhibiting hair production in a mammal. It will be appreciated by persons skilled in the art that such use may be medical and/or cosmetic in nature (as described in detail below). For example, the hair growth to be inhibited may be associated with a disease or disorder or, alternatively, may be normal hair growth (in a healthy individual) which is not desired for cosmetic reasons.

Thus, in one embodiment, the composition as defined above is for use in for treating or preventing a disease or disorder associated with unwanted and/or excessive hair growth in a mammal, such as hirsutism.

For example, the disease or disorder associated with unwanted and/or excessive hair production may be selected from the groups consisting of polycystic ovary syndrome (PCOS), the most common cause, congenital adrenal hyperplasia, Cushing's disease, growth hormone excess (acromegaly), tumours in the ovaries, adrenal gland cancer, Von Hippel-Lindau disease, insulin resistance, stromal hyperthecosis (SH), obesity, porphyria cutanea tarda, side effects of medication (such as tetrahydrogestrinone, phenytoin, minoxidil), hormonal treatment and hormonal disorders.

In a further embodiment, the composition is for treating or preventing ingrown hair in a mammal (e.g. associated with shaving, waxing and/or acne).

A further, related aspect of the invention provides a composition as defined above in the preparation of a medicament for treating or preventing a disease or disorder associated with unwanted and/or excessive hair production in a mammal in a mammal, such as hirsutism.

For example, the disease or disorder associated with unwanted and/or excessive hair production may be selected from the groups consisting of polycystic ovary syndrome (PCOS), the most common cause, congenital adrenal hyperplasia, Cushing's disease, growth hormone excess (acromegaly), tumours in the ovaries, adrenal gland cancer, Von Hippel-Lindau disease, insulin resistance, stromal hyperthecosis (SH), obesity, porphyria cutanea tarda, side effects of medication (such as tetrahydrogestrinone, phenytoin, minoxidil), hormonal treatment and hormonal disorders.

In a further embodiment, the medicament is for treating or preventing ingrown hair in a mammal (e.g. associated with shaving, waxing and/or acne).

Advantageously, the mammal is human.

The mammal, e.g. human, may be male or female.

A further, related aspect of the invention provides method for treating or preventing a disease or disorder associated with unwanted and/or excessive hair production in a mammal, such as hirsutism, the method comprising administering to the mammal an effective amount of a composition as defined above.

For example, the disease or disorder associated with unwanted and/or excessive hair production may be selected from the groups consisting of polycystic ovary syndrome (PCOS), the most common cause, congenital adrenal hyperplasia, Cushing's disease, growth hormone excess (acromegaly), tumours in the ovaries, adrenal gland cancer, Von Hippel-Lindau disease, insulin resistance, stromal hyperthecosis (SH), obesity, porphyria cutanea tarda, side effects of medication (such as tetrahydrogestrinone, phenytoin, minoxidil), hormonal treatment and hormonal disorders.

In a further embodiment, the method is for treating or preventing ingrown hair in a mammal (e.g. associated with shaving, waxing and/or acne).

The polypeptide compositions of the invention are administered to the patient in an effective amount. A ‘therapeutically effective amount’, or ‘effective amount’, or ‘therapeutically effective’, as used herein, refers to that amount which provides an inhibitory effect on hair growth. This is a predetermined quantity of active material calculated to produce the desired therapeutic effect. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art.

It will be appreciated by persons skilled in the art that the compositions of the first aspect of the invention are not limited to medical uses but may also be used as cosmetic agents (in the sense that they do not provide any physical health improvement, as such, but merely provide an aesthetic benefit to the mammal).

Thus, a fourth aspect of the invention provides the use of a composition as defined above for cosmetic hair removal in a human.

In one embodiment, the human is male. For example, the hair may be removed from an area of the body selected from the group consisting of face (e.g. cheeks, chin and above the upper lip), chest, shoulders, neck and back.

In an alternative embodiment, the human is female. For example, the hair may be removed from an area of the body selected from the group consisting of face (e.g. cheeks, chin and above the upper lip), back, legs, arms, fingers, feet, toes and pubis.

A further, related aspect of the invention provides a method for cosmetic hair removal in a human comprising administering to the human an effective amount of a composition as defined above.

In one embodiment, the human is male. For example, the hair may be removed from an area of the body selected from the group consisting of face (e.g. cheeks, chin and above the upper lip), chest, shoulders, neck and back.

In an alternative embodiment, the human is female. For example, the hair may be removed from an area of the body selected from the group consisting of face (e.g. cheeks, chin and above the upper lip), back, legs, arms, fingers, feet, toes and pubis.

The compositions of the invention may be used on their own or in combination with other therapeutic or cosmetic treatments. For example, the compositions of the invention may be used in a combination therapy with one or more of the following hair removal/prevention treatments:
shaving, plucking, depilation, hot waxing, laser hair removal, electrolysis (galvanic or thermolytic), topical creams (such as eflornithine) and/or oral medications (such as an oral contraceptives for women, anti-androgens, finasteride, and gonadotrophin-releasing hormones).

It will be further appreciated by skilled persons that the compositions of the invention may be used in vivo, ex vivo or in vitro.

Thus, in addition to being applied or administered directly to a mammal, the compositions may be used to inhibit hair production ex vivo, for example in a skin explant prior to grafting of the skin on to the mammal.

Alternatively, the polypeptide may be used for inhibiting hair growth follicles (or stem cell precursors of the same) in vitro.

Preferred, non-limiting examples which embody certain aspects of the invention will now be described, with reference to the following figures:

Preferences and options for a given aspect, feature or parameter of the invention should, unless the context indicates otherwise, be regarded as having been disclosed in combination with any and all preferences and options for all other aspects, features and parameters of the invention. For example, in one embodiment the invention provides a topical composition comprising a polypeptide of SEQ ID NO:1 to treat unwanted hair growth associated with the side effects of medication.

The listing or discussion of an apparently prior-published document in this specification should not necessarily be taken as an acknowledgement that the document is part of the state of the art or is common general knowledge.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the above description and the accompanying drawings. It should be understood, however, that the above description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

The following drawings form part of the present specification and are included to further demonstrate certain aspects of the present invention. The invention may be better understood by reference to one or more of these drawings in combination with the detailed description of specific embodiments presented herein.

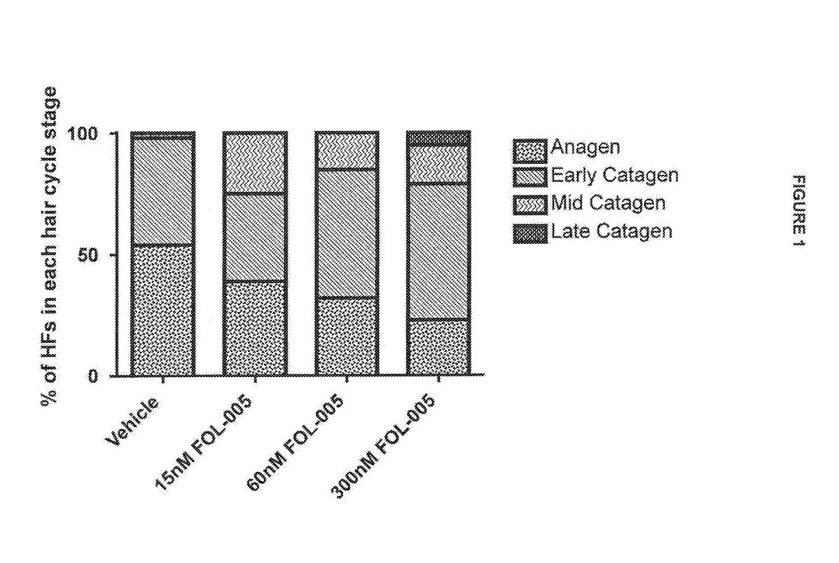

FIG. 1). Induction of anagen to catagen phase transition in cultured isolated human hair follicles by an exemplary polypeptide of the invention, “FOL-005” (SEQ ID NO: 1; data from 3 patients pooled). Number of analysed hair follicles is between 32-45 per group.

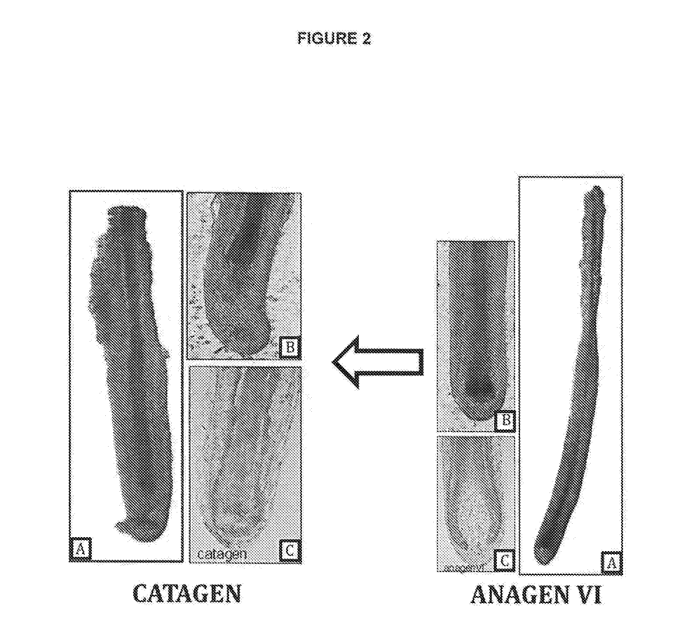

FIG. 2). Representative cryosection photographs showing FOL-005 induces transition from anagen into catagen phase
A: photo of a representative hair shaft
B: magnification of the bulb area
C: staining of the bulb area demonstrating strong effect on the dermal papilla

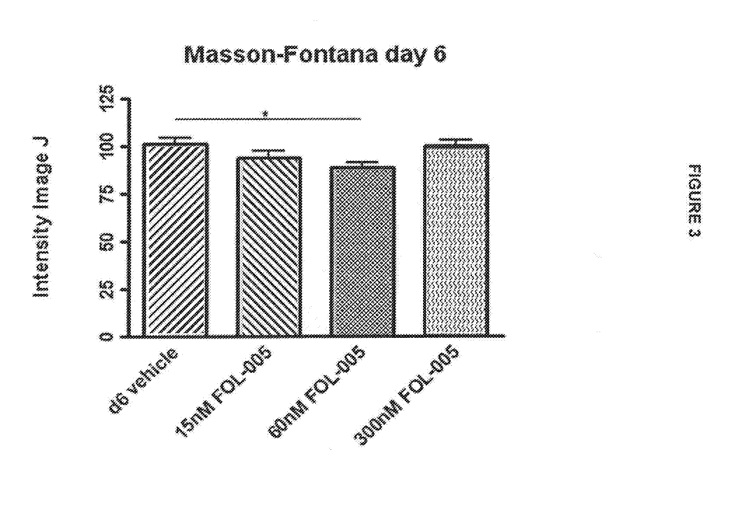

FIG. 3). Effect of FOL-005 on melatonin content in cultured isolated human hair follicles. The analysis was performed using Masson Fontana histochemistry and morphometric evaluation of each tissue section. Results presented as mean+/−SEM, n=26-47 hair follicles evaluated in each group. One-way ANOVA and Bonferroni's post-hoc test, *p<0.05

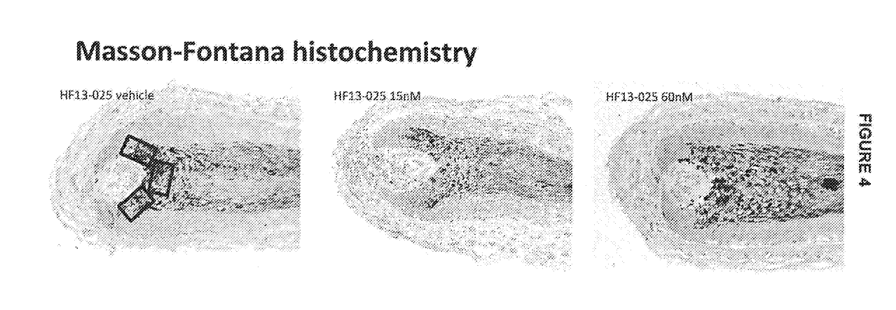

FIG. 4). Representative cryosection photographs (taken at magnification ×200) showing effect of FOL-005 on melanin pigmentation in cultured isolated human hair follicles. The black squares in the left panel indicated the morphometry reference area and the morphometry data is presented in FIG. 3.

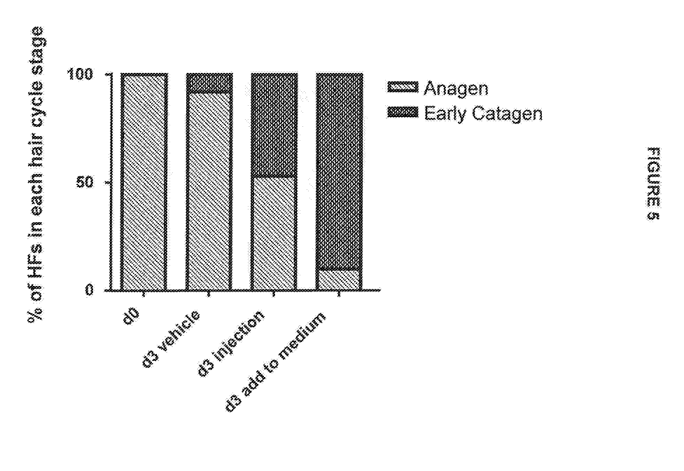

FIG. 5). Induction of anagen to catagen phase transition in intact human scalp skin by FOL-005.

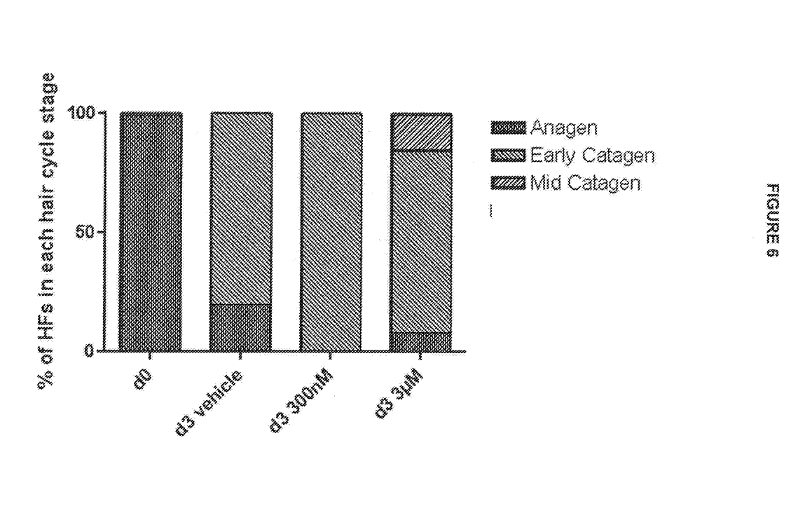

FIG. 6). Confirmation that FOL-005 induces catagen in intact human scalp skin at higher concentrations (mean of 2 patients).

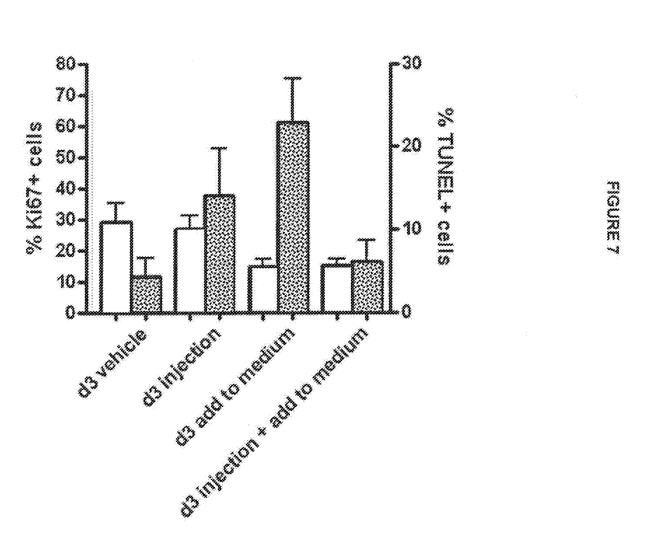

FIG. 7). Effect of FOL-005 on proliferation of Ki67 positive cells in intact human scalp skin. The concentration of FOL-005 was 60 nM, Mean+/−SEM, n=6-17 photos/punch was evaluated. “D3”=3 days after treatment.

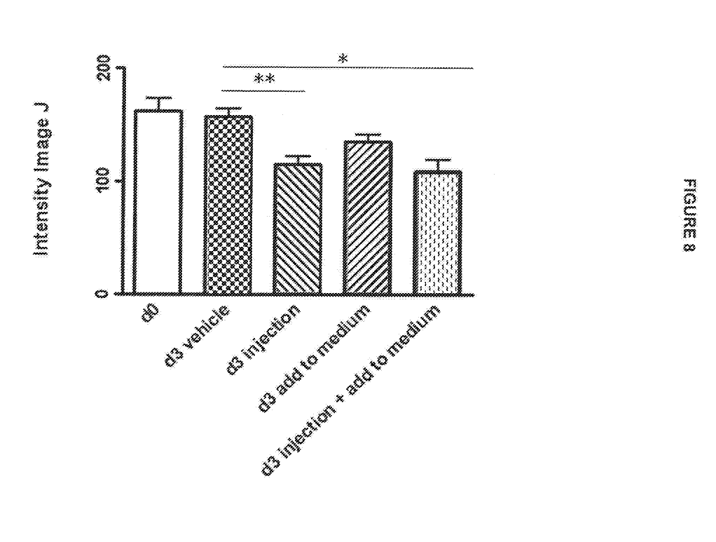

FIG. 8). Effect of FOL-005 on melatonin content in intact human scalp skin. The analysis was performed using Masson Fontana histochemistry and morphometric evaluation of each tissue section. Results presented as mean+/−SEM. One-way ANOVA and Bonferroni's post-hoc test, **p<0.01, *p<0.05. “D3”=3 days after treatment.

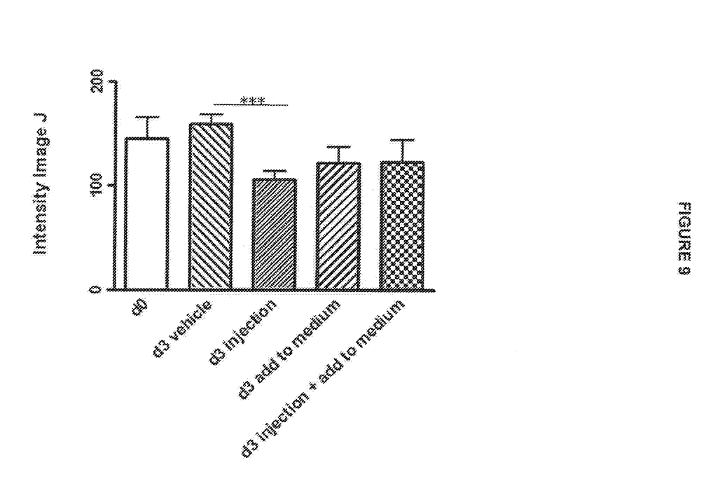

FIG. 9). Effect of FOL-005 on melanin pigmentation in an individual representative patient. ***p<0.001.

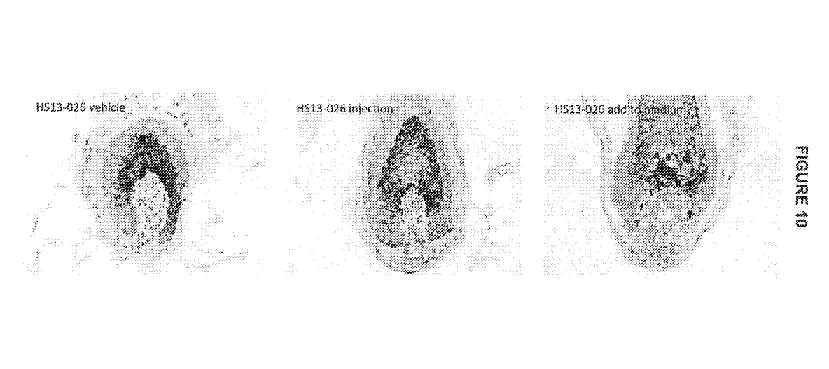

FIG. 10). Representative cryosection photographs (taken at magnification ×200) showing effect of FOL-005 on melanin pigmentation in intact human scalp skin. Photos taken of papillary dermis, 2-5 photos/punch was evaluated.

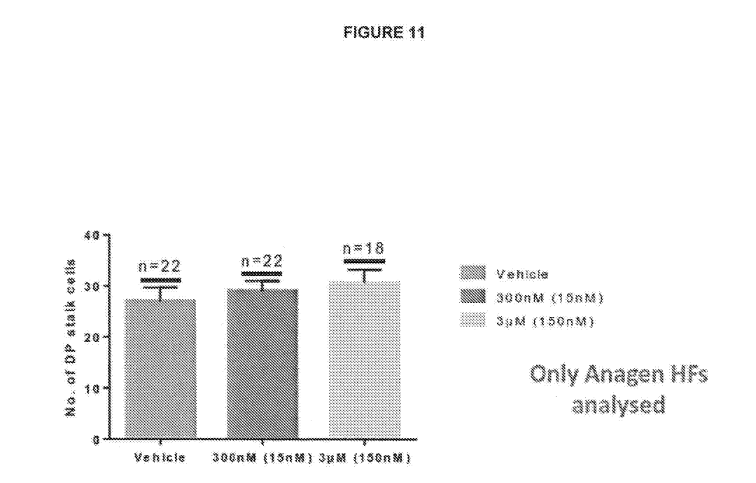

FIG. 11). Effect of FOL-005 on emigration of Dermal Papilla (DP) cells. Only Anagen Hair follicles (HFs) analysed. Results based on pooled data from 6 patients. No significant change in the number of emigrating DP cells in HFs treated with FOL-005. Mean+/−SEM, n=18-22 HFs evaluated, Mann-Whitney test, ns

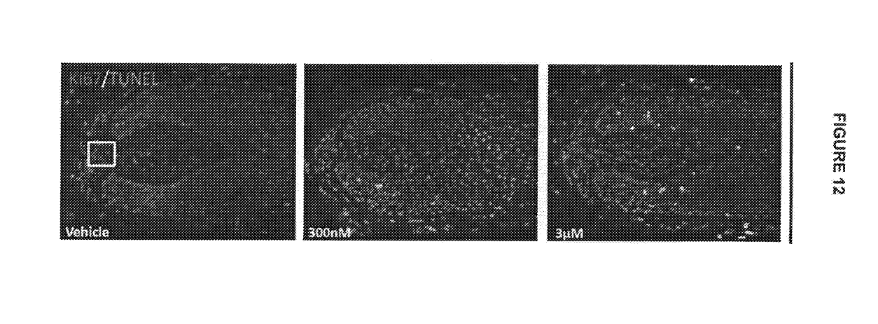

FIG. 12). Effect of FOL-005 on emigration of Dermal Papilla (DP) cells. Only Anagen Hair follicles (HFs) analysed. Results based on pooled data from 6 patients. No significant change in the number of emigrating DP cells in HFs treated with FOL-005. Mean+/−SEM, n=18-22 HFs evaluated, Mann-Whitney test, ns

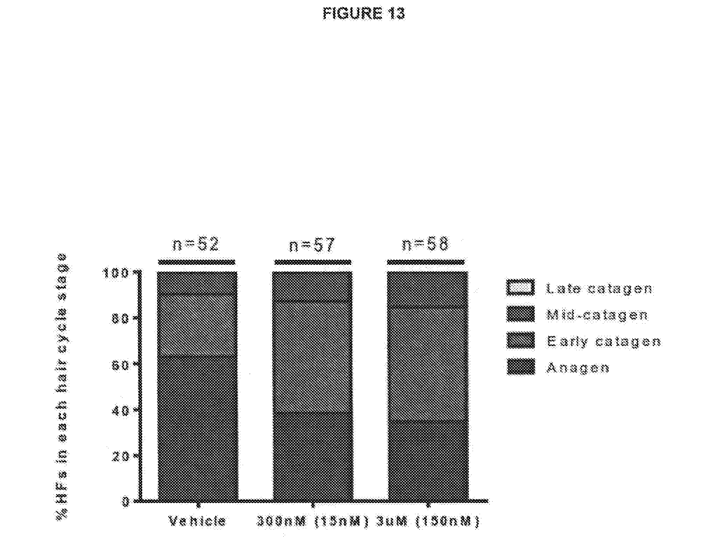

FIG. 13). Hair Cycle staging. Treatment of HFs with FOL-005 consistently induces anagen HFs within human scalp skin to enter catagen prematurely in all six patients tested (in each vertical bar, the top region corresponds to mid-catagen, the middle region to early catagen and the bottom region to anagen). Pooled data from 6 patients.

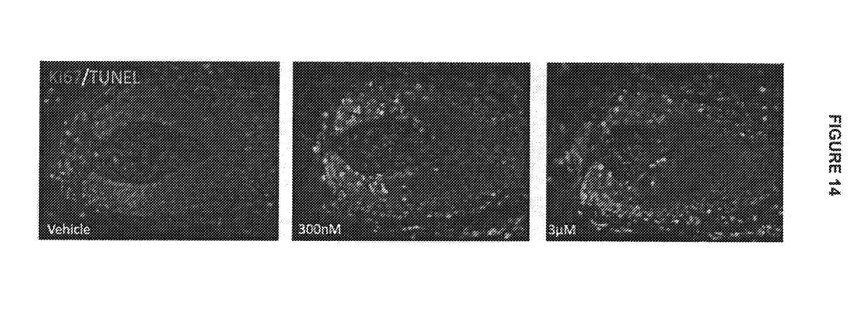

FIG. 14). Hair Cycle staging. Treatment of HFs with FOL-005 consistently induces anagen HFs within human scalp skin to enter catagen prematurely in all six patients tested. Pooled data from 6 patients.

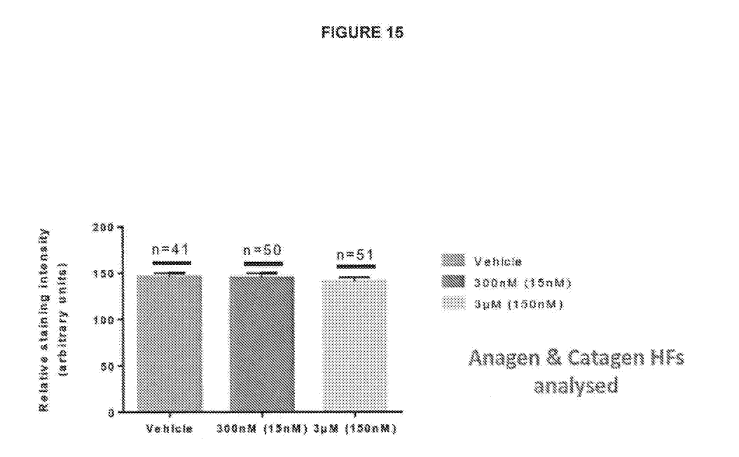

FIG. 15). Masson Fontana staining. No significant changes in HF pigmentation in HFs treated with FOL-005. Anagen & Catagen Hair Follices analysed. Pooled data from 6 patients. Mean+/−SEM, n=41-51 HFs evaluated, unpaired Student's t-test, ns

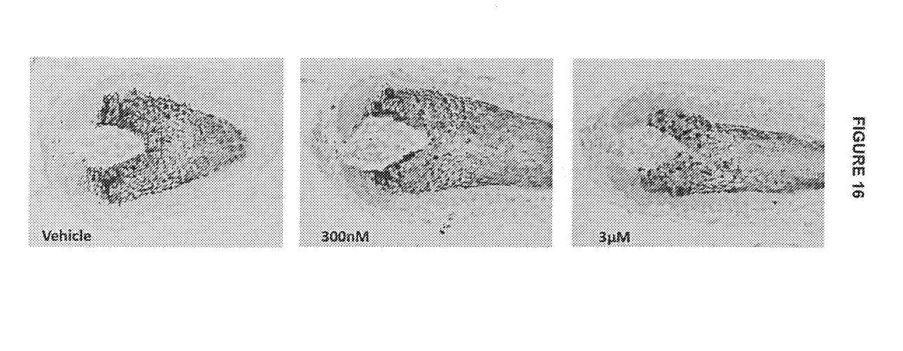

FIG. 16). Masson Fontana staining. No significant changes in HF pigmentation in HFs treated with FOL-005. Anagen & Catagen Hair Follices analysed. Pooled data from 6 patients. Mean+/−SEM, n=41-51 HFs evaluated, unpaired Student's t-test, ns

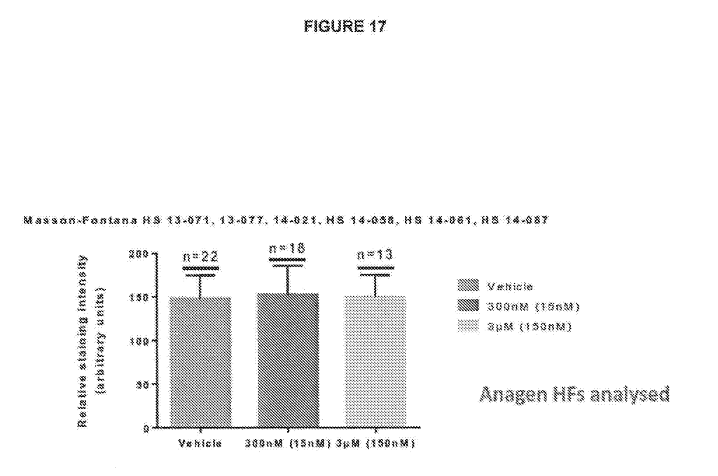

FIG. 17). Masson Fontana staining. No significant changes in HF pigmentation in HFs treated with FOL-005. Anagen HFs analysed. Pooled data from 6 patients. Mean+/−SEM, n=13-22 HFs evaluated, unpaired Student's t-test, ns

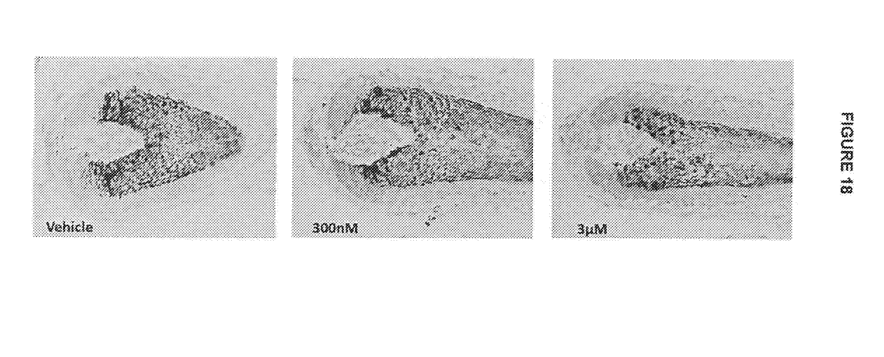

FIG. 18). Masson Fontana staining. No significant changes in HF pigmentation in HFs treated with FOL-005. Anagen HFs analysed. Pooled data from 6 patients. Mean+/−SEM, n=13-22 HFs evaluated, unpaired Student's t-test, ns

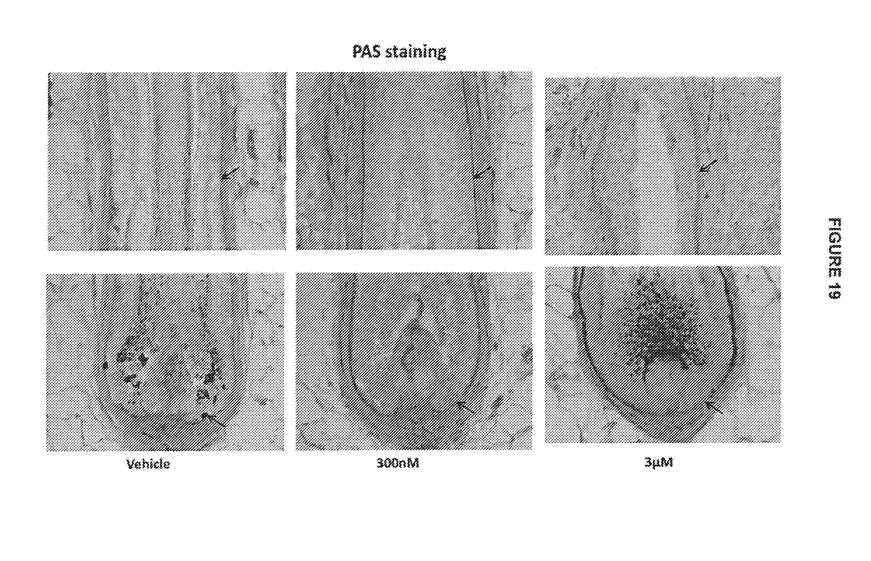

FIG. 19). No observable difference in the morphology of the basal membrane in hair follicles treated with FOL-005 (PAS staining).

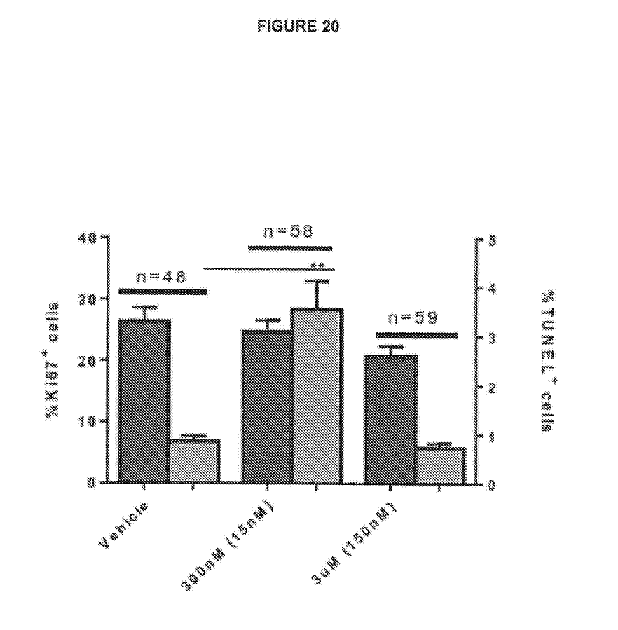

FIG. 20). Ki67/TUNEL. Significant increase in the number of apoptotic cells in HFs treated with 15 nM FOL-005 (see right-hand bar of middle pair). No significant changes were determined in the number of proliferative cells in HFs treated with FOL-005 (see left-hand column of each pair). Anagen & Catagen HFs analysed. Pooled data from 6 patients. Mean+/−SEM, n=48-59 HFs evaluated, Mann-Whitney test, *p<0.02. To reduce data distortion by outliers, Grubb's test was performed and the outlier value was eliminated from analysis.

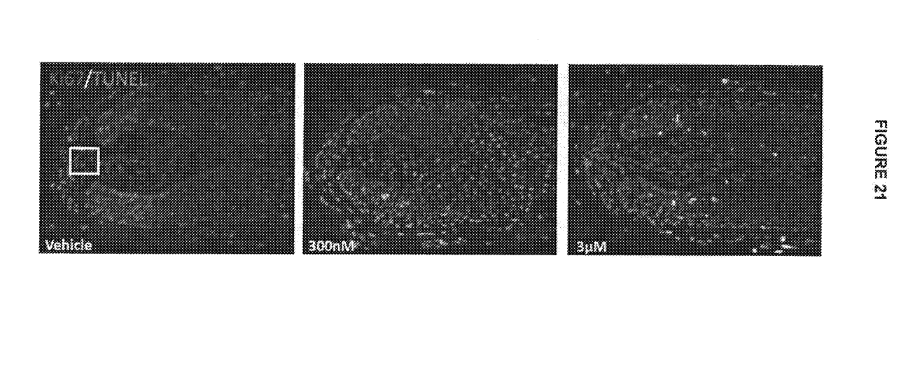

FIG. 21). Ki67/TUNEL. Significant increase in the number of apoptotic cells in HFs treated with 15 nM FOL-005. No significant changes were determined in the number of proliferative cells in HFs treated with FOL-005. Anagen & Catagen HFs analysed. Pooled data from 6 patients. Mean+/−SEM, n=48-59 HFs evaluated, Mann-Whitney test, *p<0.02. To reduce data distortion by outliers, Grubb's test was performed and the outlier value was eliminated from analysis.

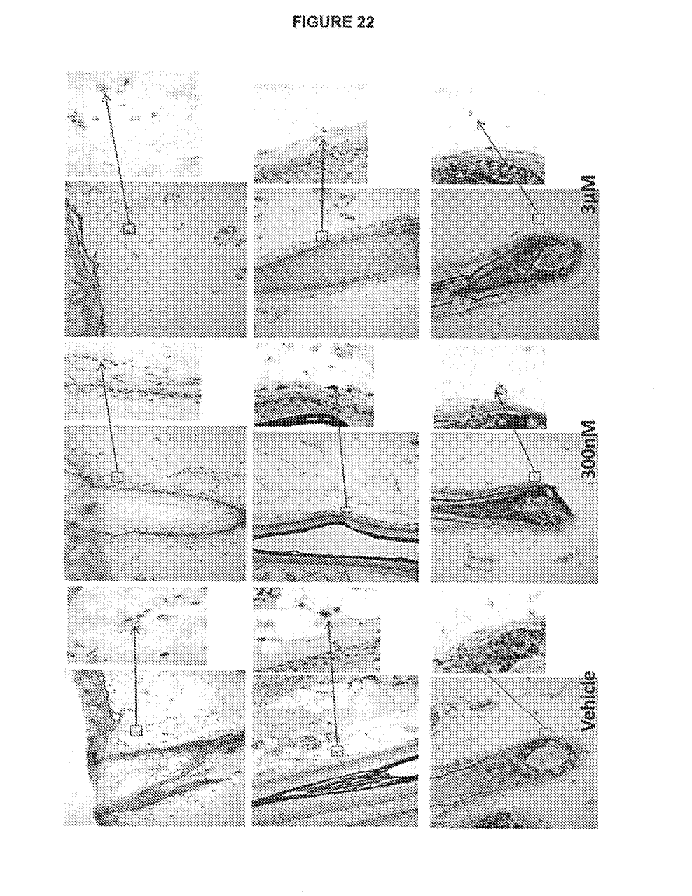

FIG. 22). Toluidine blue staining for detecting Mast cells. No marked difference in the number of histochemically detectable perifollicular mast cells.

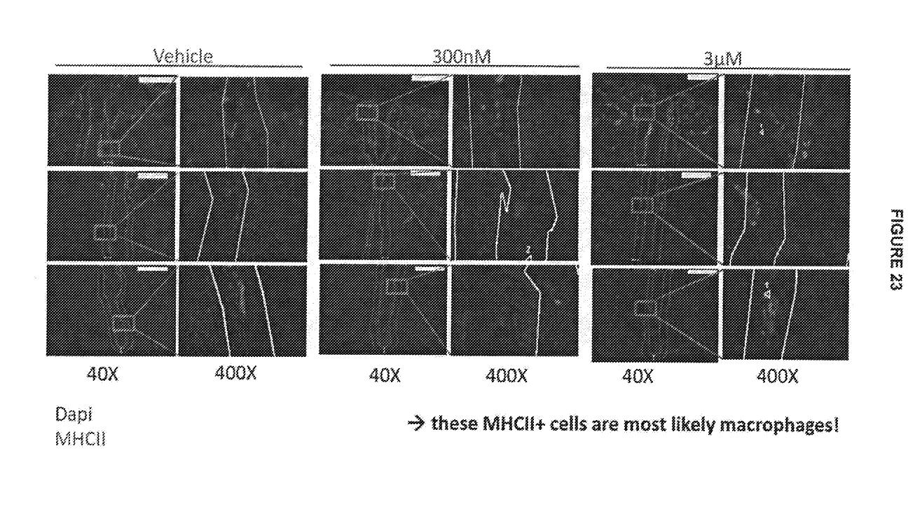

FIG. 23). MHCII staining HS 14-061 (indirect immunofluorescence. Mouse anti human Ab HLA-DP-DQ-DR from DAKO). No marked difference in the number of immunohistologically detectable perifollicular MHC class II+ cells. These MHCII+ cells are most likely macrophages.

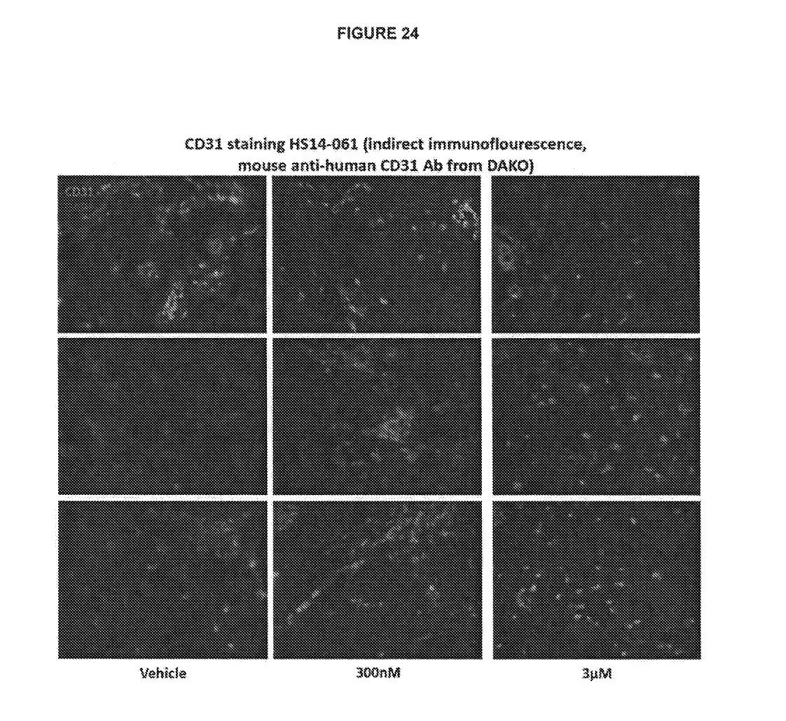

FIG. 24). CD31 staining HS14-061 (indirect immunoflourescence, mouse anti-human CD31 Ab from DAKO). No marked differences in the number of CD31+ cells (endothelial cells).

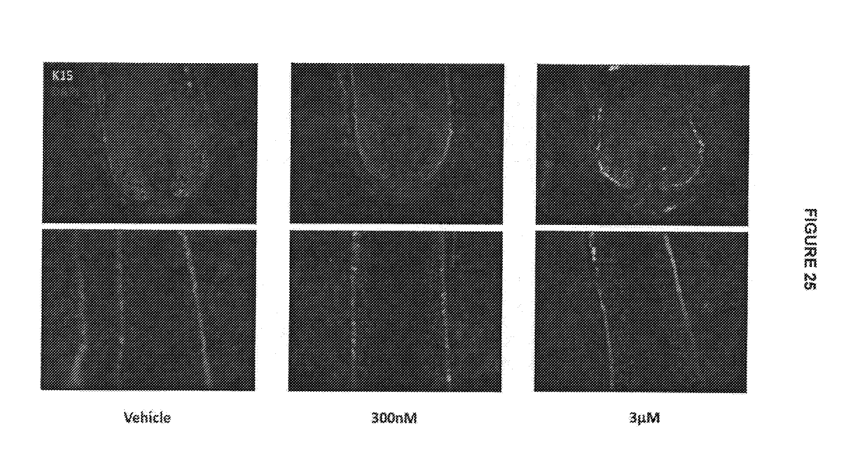

FIG. 25). K15 staining HS14-087 (indirect immunoflourescence, mouse anti-human CK15 Ab from Chemikon) No marked differences in the number of K15+ cells. Human hair follicle (HF) epithelial stem cells are not affected negatively. Preservation of HF stem cell pool and thus regenerative potential by FOL-005.

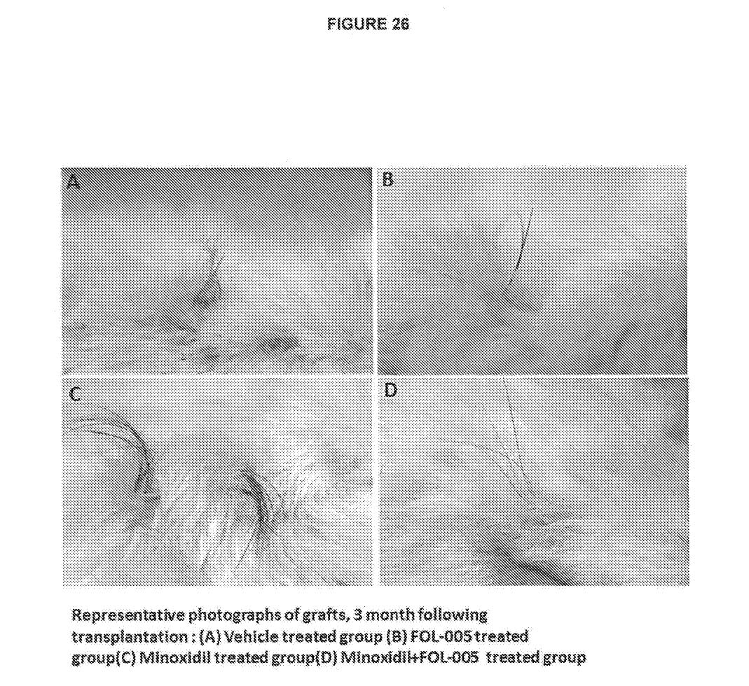

FIG. 26). Representative photographs of grafts taken three after transplantation of human hair follicles. (A) Vehicle-treated animal, (B) FOL-005-treated animal, (C) Minoxidil-treated animal and (D) FOL-005 plus minoxidil-treated animal.

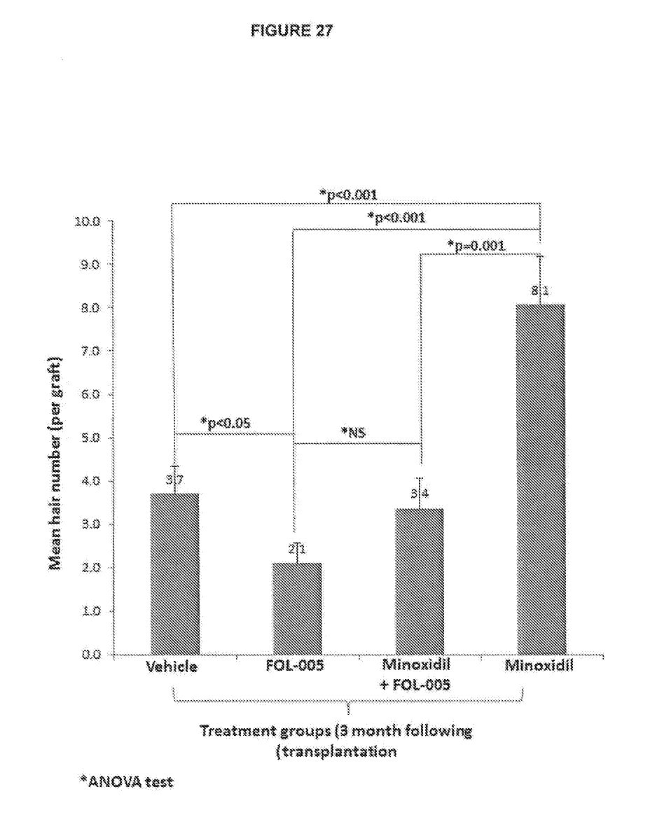

FIG. 27). Effect on mean number of human hair follicles following transplantation. (A) Vehicle-treated group, (B) FOL-005-treated group, (C) Minoxidil-treated group and (D) FOL-005 plus minoxidil-treated group.

### EXAMPLES

### Example A: Effects of Exemplary Peptide FOL-005 [SEQ ID NO:1] on Isolated Human Hair Follicles

(i) FOL-005 Induces a Change from Anagen to Catagen Phase

### Material & Methods

Anagen VI hair follicles (see Kloepper et al., 2010, the disclosures of which are incorporated herein by reference) were micro dissected from normal human scalp skin obtained with informed patient consent from three healthy adult females undergoing routine face-lift cosmetic surgery.

Isolated hair follicles were organ-cultured for 6-10 days in insulin- and hydrocortisone-supplemented William's E medium according to Philpott et al. (see Philpott et al., 1990; Bodó et al., 2009; Gáspár et al., 2010, the disclosures of which are incorporated herein by reference).

Treatment with the exemplary polypeptide FOL-005 was initiated immediately upon culturing the hair follicles, and lasted 7 to 10 days.

At the end of organ-culture, hair follicles were embedded and processed for longitudinal cryosections (Bodó et al., 2009; Gáspár et al., 2010).

### Results

Exemplary peptide of the invention FOL-005 (SEQ ID NO:1; 15 nM, 60 nM and 300 nM) induced a dose-dependent transition from anagen to catagen phase in isolated human hair follicles (see FIG. 1).

Analysis of cryosections revealed that FOL-005 promotes hair follicle regression, which indicates that FOL-005 is a strong inhibitor of human hair growth in vitro (see FIG. 2).

### (ii) FOL-005 Induces Pigmentation Changes

### Materials & Methods

Masson Fontana staining: Cryosections were air dried and fixed in ethanol-acetic acid. The sections were washed in tris-buffered saline (TBS) and distilled water several times. Cryosections were treated with ammoniacal silver solution (Fluka, Seelze, Germany) for 40 min at 56° C. in the dark. After washing in distilled water, the sections were treated with 5% aqueous sodium thiosulphate (Merck, Darmstadt, Germany) for 1 min. Next, the sections were washed in running tap water for 3 min and were counter-stained in 0.5% aqueous neutral red (Sigma). After washing in distilled water, sections were dehydrated and mounted in Eukitt (O. Kindler, Freiburg, Germany).

Immunohistochemistry: To compare proliferation and apoptosis of HFs in the different hair cycle stages double immunolabelling of Ki-67 mouse anti-Ki-67 antiserum (DAKO, Hamburg, Denmark) and TUNEL (ApopTag Fluorescein In Situ Apoptosis detection kit; Millipore, Berlin, Germany) was performed as described previously (see Kloepper et al., 2010, Experimental Dermatology 19:305-12, he relevant disclosures of which are incorporated by reference).

### Results

Melanin pigmentation analysis results are shown in FIG. 3.

At a dose of 60 nM FOL-005, a significant reduction in pigmentation was detectable, confirming an induced transition from anagen to catagen phase in the isolated hair follicles.

Representative cryosection photographs showing effect of FOL-005 on melanin pigmentation in one subject are shown in FIG. 4. In the vehicle-treated control group, more intense pigmentation is observed close to the dermal papilla, whereas following exposure to 15 nM and especially 60 nM FOL-005 the pigmentation is seen more to the right in the figure (consistent with the movement of the hair shaft as no more melanin is synthesised near the dermal papilla).

### Example B: Effects of Exemplary Peptide FOL-005 (SEQ ID NO:1) on Human Skin Pieces

(i) FOL-005 Induces a Change from Anagen to Catagen Phase

### Material & Methods

Full-thickness human scalp skin organ culture was performed for six days under serum-free conditions as described in (Lu et al., 2007, the disclosures of which are incorporated herein by reference).

In brief, scalp skin of female patients was obtained from routine face-lift surgery after informed consent. Human scalp skin was washed in William's E medium (Biochrom, Cambridge, UK) supplemented with 100 IU/ml penicillin G (Sigma, St Louis, Mo., USA), 10 μg/ml streptomycin (Sigma), 0.25 μg/ml amphotericin B (Gibco, Karlsruhe, Germany) for 5 min. The out-growing hair shafts were shaved off to the level of the epidermis; 2 mm biopsies of intact scalp skin were then punched out (parallel to the direction of hair growth, i.e. at an oblique angle), using an Acu-puncher (STIEFEL, Offenbach am Main, Germany).

The punches of human scalp tissue were carefully placed into William's E medium [supplemented with 100 IU/ml penicillin/10 μg/ml streptomycin, 10 μg/ml of insulin (Sigma), 10 ng/ml of hydrocortisone (Sigma) and 2 mmol/I of I-glutamine (Invitrogen, Paisley, UK)]. The skin fragments were left to float in the medium, with the epidermis up at air/liquid interface and the dermis/subcutis down. The cultures were maintained at 37° C. in a gassed incubator with 95% air and 5% CO₂. The culture medium was changed every other day.

Immediately after commencement of culturing, the skin fragments were exposed to the exemplary polypeptide FOL-005 (at a dose of 15 nM to 3 μM), which lasted for the duration of culturing.

At different time points, skin specimens were embedded in O.C.T.™ Tissue-TEK (Sakura, Zoeterwoude, the Netherlands), frozen in liquid nitrogen, and 6 μm cryosections were cut. The sections were then post-fixed in acetone, air-dried and processed for haematoxylin and eosin (H&E; Sigma), Giemsa or Masson-Fontana histochemistry. Individual hair follicles were analysed with respect to their stage of cycling as described (Whiting et al. 1999), while hair follicle melanization was assessed by standard silver nitrate histochemistry (Masson-Fontana stain). Quantification of well-defined reference areas within the proximal hair matrix was performed using the Image J software (NIH).

To evaluate apoptotic and proliferating cells, double immunofluorescence was applied. Terminal dUTP nicked label-ling (ApopTag®Fluorescein in situ Apoptosis Detection Kit; Chemicon, Tenecula, Calif., USA) was used as a marker for apoptosis, and Ki67 immunoreactivity (DakoCytomation, Glostrup, Denmark) as indicator of cell proliferation, as previously described (Foitzik et. al., 2006).

### Results

FOL-005 induced a rapid transition from anagen to catagen phase in intact human scalp skin (see FIGS. 5 and 6).

Almost all hair follicles are observed in early catagen following addition of FOL-005 to the culture medium.

Catagen was also induced by injection of FOL-005 into the scalp skin tissue.

Analysis of cryosections revealed that FOL-005 promotes hair follicle regression, which indicates that FOL-005 is a strong inhibitor of human hair growth in vitro (see FIG. 2).

### (ii) FOL-005 Inhibits the Proliferation of Ki67 Positive Cells and Increases Apoptosis

### Materials & Methods

Masson Fontana staining and immunohistochemistry was performed as described above.

The proliferation of Ki67 positive cells and apoptosis therein was determined as described in Whiting et al., 1999, J Investig Dermatol Symp Proc 4:282-284 (the relevant disclosures of which are incorporated by reference).

Cells were exposed to exemplary polypeptide FOL-005 at a dose of 60 nM.

### Results

There is a strong tendency towards reduced proliferation in the FOL-005 treated skin as represented by a reduced proportion of Ki67 positive cells (see FIG. 7; white bar).

In keeping with an inhibition of hair growth, there is also an increased level of apoptosis as indicated by the proportion of TUNEL positive cells following FOL-05 treatment (see FIG. 7; shaded bar).

(iii) FOL-005 Induces Pigmentation Changes

### Materials & Methods

Pigmentation changes in isolated follicles treated with exemplary polypeptide FOL-005 were assessed by Masson-Fontana histochemistry (see Whiting et al., 1999, J Investig Dermatol Symp Proc 4:282-284, the relevant disclosures of which are incorporated by reference).

### Results

Melanin pigmentation analysis results are shown in FIGS. 8 and 9.

Exposure of intact human scalp skin to FOL-005, either by injection or addition to the culture medium, induced a significant reduction in pigmentation, confirming a transition from anagen to catagen phase in the hair follicles.

Representative cryosection photographs showing effect of FOL-005 on melanin pigmentation in one subject are shown in FIG. 10. The vehicle-treated control group (left panel) shows strong pigmentation, in contrast to the FOL-005 treated hair follicles which display less intense pigmentation (middle and right panel).

### Example C: Ex Vivo Assessment of FOL-005 [SEQ ID NO:1] on Scalp Hair Follicles (HFs)

The above ex vivo assessment of the effect of exemplary peptide FOL-005 on scalp hair follicles (HFs) was repeated in order to demonstrate the robustness of the observed inhibition of hair growth.

The following experiments were performed using human HFs in organ-cultured full-thickness human scalp skin fragments (4 mm, 6 female patients, 42-65 yrs).

### a) Emigration of DP Cells

No significant effect of FOL-005 treatment was observed on the number of emigrating dermal papilla (DP) cells in HFs (pooled data from six patients; mean+/−SEM, n=18-22 HFs, Mann-Whitney test).

Results are shown in FIGS. 11 and 12.

b) Analysis of HF cycling Treatment of HFs with FOL-005 consistently induced anagen HFs within human scalp skin to enter catagen prematurely in all six patients tested (pooled data from six patients).

Results are shown in FIGS. 13 and 14.

### c) Hair Pigmentation

No significant changes in HF pigmentation were observed in HFs treated with FOL-005 at the doses tested (mean+/−SEM, unpaired Student's t-test).

Results are shown in FIGS. 15 and 16 (anagen and catagen HFs analysed).

Further results are shown in FIGS. 17 and 18 9 (anagen HFs only analysed).

### d) Hair Follicle Morphology (PAS)

No observable difference was observed in the morphology of the BM in HFs treated with FOL-005 (indicative of an absence of any toxic effect)

Results are shown in FIG. 19.

### e) Proliferation/Apoptosis

A significant increase was observed in the number of apoptotic cells in HFs treated with 15 nM FOL-005 (anagen and catagen HFs analysed).

No significant changes were determined in the number of proliferative cells in HFs treated with FOL-005 at this dose.

Mean+/−SEM, n=48-59 HFs evaluated, Mann-Whitney test, *p<0.02. To reduce data distortion by outliers, Grubb's test was performed and the outlier value was eliminated from analysis.

Results are shown in FIGS. 20 and 21.

### f) Stain for MCs (Mast Cells), MHCII+, CD31+ Cells

No marked difference was observed in the number of histochemically-detectable perifollicular mast cells (see FIG. 22; Toluidine blue staining for detecting Mast cells)

No marked difference was observed in the number of immunohistologically-detectable perifollicular MHC class II+ cells (see FIG. 23)

No marked differences in the number of CD31+ cells (endothelial cells) (see FIG. 24).

### g) Stain for K15+ Cells

No marked differences were observed in the number of K15+ cells

K15 staining was performed using HS14-087 (indirect immunoflourescence, mouse anti-human CK15 Ab from Chemikon).

Human HF epithelial stem cells were not affected negatively.

Preservation of the HF stem cell pool was achieved thus demonstrating the regenerative potential by FOL-005 (see FIG. 25)

### Summary

The above findings confirm and extend the results in Example B showing that exemplary peptide FOL-005 robustly promotes catagen development in human scalp HFs, by inducing the following effects.
(a) an increased % of catagen HFs after treatment with FOL-005;
(b) an increase in apoptotic cells in the hair matrix of treated HFs in pooled data; and
(c) a tendency towards increased emigration of DP from anagen HFs after treatment

The exemplary peptide FOL-005 appears well-tolerated, as evidenced by the following observations:
(a) No significant change in HF melanin content after treatment with FOL-005;
(b) No marked differences in basement membrane morphology or in the number of perifollicular macrophages and mast cells and of CD31+ cells in FOL-005-treated samples; and
(c) No marked difference in the number of K15+ cells

### Conclusions

FOL-005 clearly promotes HF catagen, confirming its activity as a human hair growth inhibitor: it appears well-tolerated & effective. There were no signs of any toxic effects.

### Example D: Effects of Exemplary Peptide FOL-005 [SEQ ID NO:1] on Human Skin Pieces

The effect of FOL-005 on hair growth of human male scalp skin grafted onto SCID mice was studied.

The aim of the study was to investigate the effect of FOL-005 on hair growth of human male scalp skin with tendency to androgenetic alopecia. In order to address this issue, fort-two female SCID/beige mice, six weeks of age, were involved in the study. Punch grafts, 3 mm², obtained from scalp skin of human volunteers with tendency to develop common alopecia, were transplanted onto SCID/beige mice (three grafts per mouse).

One week following transplantation, the mice were randomly divided into four treated groups as follows:
1. Vehicle (10 mice) was injected intradermally twice a week and served as a negative control.
2. Minoxidil (5%) (11 mice) was applied topically twice a day and served as appositive control.
3. FOL-005 (300 nM per graft) (11 mice) was injected intradermally twice a week.
4. Minoxidil 5%+FOL-005/Minoxidil (5%) (11 mice) was applied topically twice a day. FOL-005 (300 nM per graft) was injected intradermally twice a week. For the first 4 weeks the animals were treated with minoxidil only (in order to initiate hair growth) and then for the remaining period (2 months) FOL-005 treatment was added as injections, while minoxidil treatment was maintained as a topical application.

The number of hairs/graft was counted twice a week by two independent observers. Three month following skin transplantation, the grafts were obtained from the mice and were snapped frozen in liquid nitrogen and stored in −80° C. for further analysis.

A significant decrease in the number of hairs/graft was observed in the FOL-005 treated group (2.1±0.4) as compared to the vehicle one (3.7±0.6 respectively, p<0.05) (see FIG. 26 and FIG. 27).

Furthermore, a significant decrease in the number of hairs/graft was detected in the FOL-005+Minoxidil (3.4±0.7) treated group as compared to group treated with Minoxidil alone (8.1±0.1, p<0.001).

A similar mean number of hairs/graft was observed in the Vehicle group and FOL-005+Minoxidil (3.7±0.6 and 3.4±0.7, p=NS).

The results of this study confirm that FOL-005 possesses an inhibitory effect on hair growth. The full hair growth-inhibitory potential of FOL-005 in human scalp skin in vivo is also underscored by the fact that it strongly antagonized the hair growth-promoting effects of minoxidil (one of the best-recognized hypertrichosis-inducing agents in clinical medicine).

### REFERENCES

Philpott M P, Sanders D A, Westgate G E, Kealey T. Human hair growth in vitro: a model for the study of hair follicle biology. Journal of Dermatological Science. 1994; 7:S55-S72.
Bodo E, Kromminga A, Biro T, Borbiro I, Gaspar E, Zmijewski M A, et al. Human female hair follicles are a direct, nonclassical target for thyroid-stimulating hormone. The Journal of investigative dermatology. 2009; 129(5):1126-39.
Gaspar E, Hardenbicker C, Bodo E, Wenzel B, Ramot Y, Funk W, et al. Thyrotropin releasing hormone (TRH): a new player in human hair-growth control. FASEB journal: official publication of the Federation of American Societies for Experimental Biology. 2010; 24(2):393-403.
Kloepper J E, Sugawara K, Al-Nuaimi Y, Gaspar E, van Beek N, Paus R. Methods in hair research: how to objectively distinguish between anagen and catagen in human hair follicle organ culture. Experimental Dermatology. 2010; 19:305-12.
Lu Z, Hasse S, Bodo E, Rose C, Funk W, Paus R. Towards the development of a simplified long-term organ culture method for human scalp skin and its appendages under serum-free conditions. Experimental Dermatology. 2007; 16(1):37-44.
Foitzik K, Krause K, Conrad F, Nakamura M, Funk W, Paus R. Human scalp hair follicles are both a target and a source of prolactin, which serves as an autocrine and/or paracrine promoter of apoptosis-driven hair follicle regression. Am J Pathol. 2006; 168(3):748-56.
Whiting D A, Waldstreicher J, Sanchez M, Kaufman K D. Measuring reversal of hair miniaturization in androgenetic alopecia by follicular counts in horizontal sections of serial scalp biopsies: results of fi-nasteride 1 mg treatment of men and postmenopausal women. J Investig Dermatol Symp Proc 1999: 4: 282-284.

1. A composition for use in inhibiting hair production in a mammal comprising:
(a) a polypeptide comprising or consisting of an amino acid sequence of SEQ ID NO:1
SEQ ID NO: 1
VDTYDGDISVVYGLR
or a fragment or variant thereof which retains an inhibitory activity on mammalian hair production; and
(b) a pharmaceutically acceptable and/or cosmetically acceptable excipient, carrier, adjuvant or diluent
wherein the polypeptide is between 5 and 50 amino acids in length.

2. A composition for use in inhibiting hair production according to claim 1 wherein the composition is capable of inhibiting the growth of human hair.

3. A composition for use in inhibiting hair production according to claim 1 or 2 wherein the composition is capable of inhibiting the production of hair by healthy skin.

4. A composition for use in inhibiting hair growth according to any one of the preceding claims wherein the composition is capable of inhibiting hair production on the scalp.

5. A composition for use in inhibiting hair production according to any one of the preceding claims wherein the composition is capable of inhibiting existing hair follicles.

6. A composition for use in inhibiting hair production according to any one of the preceding claims wherein the composition induces:
(a) a decrease in the length of the anagen phase (e.g. by inducing an anagen to catagen phase change) in hair follicles; and/or
(b) an increase in the length of the catagen phase in hair follicles; and/or
(c) an increase in the length of the telogen phase in hair follicles.

7. A composition for use in inhibiting hair production according to claim 6 wherein the composition is capable of inducing the hair follicles to switch from the production of terminal hair to vellus hair.

8. A composition for use in inhibiting hair production according to any one of the preceding claims wherein the composition is capable of inhibiting the formation of new hair follicles, or stem cells for producing the same.

9. A composition for use in inhibiting hair production according to any one of the preceding claims wherein the polypeptide is fewer than 40 amino acids in length, for example fewer than 35, 30, 28, 26, 24, 22, 20, 19, 18, 17, 16, 15, 14, 13, 12, 11, 10 or fewer amino acids in length.

10. A composition for use in inhibiting hair production according to claim 9 wherein the polypeptide is between 5 and 30 amino acids in length, for example between 5 and 20, between 5 and 20, between 8 and 20, between 8 and 16, or between 10 and 15 amino acids in length.

11. A composition for use in inhibiting hair production according to claim 10 wherein the polypeptide is between 10 and 15 amino acids in length.

12. A composition for use in inhibiting hair production according to any one of the preceding claims wherein the polypeptide consists of an amino acid sequence of SEQ ID NO:1.

13. A composition for use in inhibiting hair production according to any one of claims 1 to 11 wherein the polypeptide consists of an amino acid sequence of SEQ ID NO:2
SEQ ID NO: 2
VDTYDGDISVVYGLS

14. A composition for use in inhibiting hair production according to any one of claims 1 to 11 wherein the polypeptide consists of a fragment of the amino acid sequence of SEQ ID NO: 1.

15. A composition for use in inhibiting hair production according to claim 14 wherein the fragment is 14 or fewer amino acids in length, for example 13, 12, 11, 10, 9, 8, 7, 6 or 5 amino acids in length.

16. A composition for use in inhibiting hair production according to claim 14 or 15 wherein the fragment comprises or consists of an amino acid sequence according to any one of SEQ ID NOs: 3 to 65
(a) 14-amino acid peptides:
SEQ ID NO: 3
VDTYDGDISVVYGL
SEQ ID NO: 4
DTYDGDISVVYGLR
SEQ ID NO: 5
TYDGDISVVYGLRS
(b) 13-amino acid peptides:
SEQ ID NO: 6
VDTYDGDISVVYG
SEQ ID NO: 7
DTYDGDISVVYGL
SEQ ID NO: 8
TYDGDISVVYGLR
SEQ ID NO: 9
YDGDISVVYGLRS
(c) 12-amino acid peptides:
SEQ ID NO: 10
VDTYDGDISVVY
SEQ ID NO: 11
DTYDGDISVVYG
SEQ ID NO: 12
TYDGDISVVYGL
SEQ ID NO: 13
YDGDISVVYGLR
SEQ ID NO: 14
DGDISVVYGLRS
(d) 11-amino acid peptides:
SEQ ID NO: 15
VDTYDGDISVV
SEQ ID NO: 16
DTYDGDISVVY
SEQ ID NO: 17
TYDGDISVVYG
SEQ ID NO: 18
YDGDISVVYGL
SEQ ID NO: 19
DGDISVVYGLR
SEQ ID NO: 20
GDISVVYGLRS
(e) 10-amino acid peptides:
SEQ ID NO: 21
VDTYDGDISV
SEQ ID NO: 22
DTYDGDISVV
SEQ ID NO: 23
TYDGDISVVY
SEQ ID NO: 24
YDGDISVVYG
SEQ ID NO: 25
DGDISVVYGL
SEQ ID NO: 26
GDISVVYGLR
SEQ ID NO: 27
DISVVYGLRS
(f) 9-amino acid peptides:
SEQ ID NO: 28
VDTYDGDIS
SEQ ID NO: 29
DTYDGDISV
SEQ ID NO: 30
TYDGDISVV
SEQ ID NO: 31
YDGDISVVY
SEQ ID NO: 32
DGDISVVYG
SEQ ID NO: 33
GDISVVYGL
SEQ ID NO: 34
DISVVYGLR
SEQ ID NO: 35
ISVVYGLRS
(q) 8-amino acid peptides:
SEQ ID NO: 36
VDTYDGDI
SEQ ID NO: 37
DTYDGDIS
SEQ ID NO: 38
TYDGDISV
SEQ ID NO: 39
YDGDISVV
SEQ ID NO: 40
DGDISVVY
SEQ ID NO: 41
GDISVVYG
SEQ ID NO: 42
DISVVYGL
SEQ ID NO: 43
ISVVYGLR
(h) 7-amino acid peptides:
SEQ ID NO: 44
VDTYDGD
SEQ ID NO: 45
DTYDGDI
SEQ ID NO: 46
TYDGDIS
SEQ ID NO: 47
YDGDISV
SEQ ID NO: 48
DGDISVV
SEQ ID NO: 49
GDISVVY
SEQ ID NO: 50
DISVVYG
SEQ ID NO: 51
ISVVYGL
(i) 6-amino acid peptides:
SEQ ID NO: 52
DTYDGD
SEQ ID NO: 53
TYDGDI
SEQ ID NO: 54
YDGDIS
SEQ ID NO: 55
DGDISV
SEQ ID NO: 56
GDISVV
SEQ ID NO: 57
DISVVY
SEQ ID NO: 58
ISVVYG
(j) 5-amino acid peptides:
SEQ ID NO: 59
TYDGD
SEQ ID NO: 60
YDGDI
SEQ ID NO: 61
DGDIS
SEQ ID NO: 62
GDISV
SEQ ID NO: 63
DISVV
SEQ ID NO: 64
ISVVY
SEQ ID NO: 65
SVVYG.

17. A composition for use in inhibiting hair production according to claim 16 wherein the fragment comprises or consists of an amino acid sequence of SEQ ID NO: 26.

18. A composition for use in inhibiting hair production according to any one of the preceding claims wherein the polypeptide comprises or consists of a variant of the amino acid sequence of SEQ ID NO: 1, or of a fragment thereof.

19. A composition for use in inhibiting hair production according to claim 18 wherein the variant comprises or consists of an amino acid sequence with at least 50% identity to the amino acid sequence of SEQ ID NO: 1, more preferably at least 60%, 70% or 80% or 85% or 90% identity to said sequence, and most preferably at least 95%, 96%, 97%, 98% or 99% identity to said amino acid sequence.

20. A composition for use in inhibiting hair production according to claim 18 or 19 wherein the variant comprises or consists of an amino acid sequence of SEQ ID NO: 1, or a fragment thereof, in which one or more amino acids is conservatively substituted.

21. A composition for use in inhibiting hair production according to any one of claims 18 to 20 wherein the polypeptide comprises or consists of one or more additional amino acids, inserted at the N- and/or C-terminus and/or internally within the amino acid sequence of SEQ ID NO:1.

22. A composition for use in inhibiting hair production according to claim 21 wherein the polypeptide comprises or consists of at least 2, 3, 4, 5, 6, 7, 8, 9, 10, 15 or 20 additional amino acids.

23. A composition for use in inhibiting hair production according to claim 22 wherein the additional amino acids are the amino acids from the corresponding positions of the wildtype human osteopontin (SEQ ID NO:66).

24. A composition for use in inhibiting hair production according to any one of claim 18 or 19 wherein the polypeptide comprises or consists of an amino acid sequence of SEQ ID NO: 67, or of a fragment or variant thereof
SEQ ID NO: 67
VDTYDGRGDSVVYGLR

25. A composition for use in inhibiting hair production according to claim 24 wherein the fragment comprises 15 or fewer amino acids in length, for example 14, 13, 12, 11, 10, 9, 8, 7, 6 or 5 amino acids in length.

26. A composition for use in inhibiting hair production according to claim 24 wherein the polypeptide comprises or consists of a variant of the amino acid sequence of SEQ ID NO:67, or of a fragment thereof.

27. A composition for use in inhibiting hair production according to claim 26 wherein the variant comprises or consists of an amino acid sequence with at least 50% identity to the amino acid sequence of SEQ ID NO:67, more preferably at least 60%, 70% or 80% or 85% or 90% identity to said sequence, and most preferably at least 95%, 96%, 97%, 98% or 99% identity to said amino acid sequence.

28. A composition for use in inhibiting hair production according to claim 26 or 27 wherein the variant comprises or consists of an amino acid sequence of SEQ ID NO:67, or a fragment thereof, in which one or more amino acids is conservatively substituted.

29. A composition for use in inhibiting hair production according to claim 26 or 27 wherein the variant comprises or consists of an amino acid sequence of SEQ ID NO:67, or a fragment thereof, in which the RGD sequence is inactivated.

30. A composition for use in inhibiting hair production according to any one of claims 26 to 29 wherein the polypeptide comprises or consists of one or more additional amino acids, inserted at the N- and/or C-terminus and/or internally within the amino acid sequence of SEQ ID NO:67.

31. A composition for use in inhibiting hair production according to claim 30 wherein the polypeptide comprises or consists of at least 2, 3, 4, 5, 6, 7, 8, 9, 10, 15 or 20 additional amino acids.

32. A composition for use in inhibiting hair production according to claim 31 wherein the additional amino acids are the amino acids from the corresponding positions of the wildtype human osteopontin.

33. A composition for use in inhibiting hair production according to any one of the preceding claims wherein the polypeptide comprises or consists of the following amino acid sequence:
SEQ ID NO: 68
X1-X2-X3-X4-X5-X6-D-X8-I-X10-X11-X12-Y-G-X15-X16-
X17
wherein
X1 is V, E, G or K;
X2 is D, E, S or P;
X3 is any amino acid;
X4 is Y, P, N or A;
X5 is D, N or E;
X6 is G or I;
X8 is G or absent;
X10 is S or E;
X11 is V or L;
X12 is V, A or T
X15 is L or I;
X16 is R or K; and
X17 is R, K or absent.

34. A composition for use in inhibiting hair production according to any one of claims 18, 19 and 33 wherein the polypeptide comprises or consists of an amino acid sequence of SEQ ID NO:69 or 70, or of a fragment or variant thereof
SEQ ID NO: 69
VDVPNGDISLAYGLR
SEQ ID NO: 70
DVPNGDISLAYGLRS

35. A composition for use in inhibiting hair production according to claim 34 wherein the fragment comprises 14 or fewer amino acids in length, for example 13, 12, 11, 10, 9, 8, 7, 6 or 5 amino acids in length.

36. A composition for use in inhibiting hair production according to claim 34 or 35 wherein the fragment comprises or consists of an amino acid sequence according to any one SEQ ID NOs: 71 to 133
(a) 14-amino acid peptides:
SEQ ID NO: 71
VDVPNGDISLAYGL
SEQ ID NO: 72
DVPNGDISLAYGLR
SEQ ID NO: 73
VPNGDISLAYGLRS
(b) 13-amino acid peptides:
SEQ ID NO: 74
VDVPNGDISLAYG
SEQ ID NO: 75
DVPNGDISLAYGL
SEQ ID NO: 76
VPNGDISLAYGLR
SEQ ID NO: 77
PNGDISLAYGLRS
(c) 12-amino acid peptides:
SEQ ID NO: 78
VDVPNGDISLAY
SEQ ID NO: 79
DVPNGDISLAYG
SEQ ID NO: 80
VPNGDISLAYGL
SEQ ID NO: 81
PNGDISLAYGLR
SEQ ID NO: 82
NGDISLAYGLRS
(d) 11-amino acid peptides:
SEQ ID NO: 83
VDVPNGDISLA
SEQ ID NO: 84
DVPNGDISLAY
SEQ ID NO: 85
VPNGDISLAYG
SEQ ID NO: 86
PNGDISLAYGL
SEQ ID NO: 87
NGDISLAYGLR
SEQ ID NO: 88
GDISLAYGLRS
(e) 10-amino acid peptides:
SEQ ID NO: 89
VDVPNGDISL
SEQ ID NO: 91
DVPNGDISLA
SEQ ID NO: 91
VPNGDISLAY
SEQ ID NO: 92
PNGDISLAYG
SEQ ID NO: 93
NGDISLAYGL
SEQ ID NO: 94
GDISLAYGLR
SEQ ID NO: 95
DISLAYGLRS
(f) 9-amino acid peptides:
SEQ ID NO: 96
VDVPNGDIS
SEQ ID NO: 97
DVPNGDISL
SEQ ID NO: 98
VPNGDISLA
SEQ ID NO: 99
PNGDISLAY
SEQ ID NO: 100
NGDISLAYG
SEQ ID NO: 101
GDISLAYGL
SEQ ID NO: 102
DISLAYGLR
SEQ ID NO: 103
ISLAYGLRS
(g) 8-amino acid peptides:
SEQ ID NO: 104
VDVPNGDI
SEQ ID NO: 105
DVPNGDIS
SEQ ID NO: 106
VPNGDISL
SEQ ID NO: 107
PNGDISLA
SEQ ID NO: 108
NGDISLAY
SEQ ID NO: 109
GDISLAYG
SEQ ID NO: 110
DISLAYGL
SEQ ID NO: 111
ISLAYGLR
(h) 7-amino acid peptides:
SEQ ID NO: 112
VDVPNGD
SEQ ID NO: 113
DVPNGDI
SEQ ID NO: 114
VPNGDIS
SEQ ID NO: 115
PNGDISL
SEQ ID NO: 116
NGDISLA
SEQ ID NO: 117
GDISLAY
SEQ ID NO: 118
DISLAYG
SEQ ID NO: 119
ISLAYGL
(i) 6-amino acid peptides:
SEQ ID NO: 120
DVPNGD
SEQ ID NO: 121
VPNGDI
SEQ ID NO: 122
PNGDIS
SEQ ID NO: 123
NGDISL
SEQ ID NO: 124
GDISLA
SEQ ID NO: 125
DISLAY
SEQ ID NO: 126
ISLAYG
(i) 5-amino acid peptides:
SEQ ID NO: 127
VPNGD
SEQ ID NO: 128
PNGDI
SEQ ID NO: 129
NGDIS
SEQ ID NO: 130
GDISL
SEQ ID NO: 131
DISLA
SEQ ID NO: 132
ISLAY
SEQ ID NO: 133
SLAYG

37. A composition for use in inhibiting hair production according to claim 36 wherein the fragment comprises or consists of an amino acid sequence of SEQ ID NO:94.
SEQ ID NO: 94
GDISLAYGLR

38. A composition for use in inhibiting hair production according to claim 34 wherein the polypeptide comprises or consists of a variant of the amino acid sequence of SEQ ID NO: 69 or 70, or of a fragment thereof.

39. A composition for use in inhibiting hair production according to claim 38 wherein the variant comprises or consists of an amino acid sequence with at least 50% identity to the amino acid sequence of SEQ ID NO: 69 or 70, more preferably at least 60%, 70% or 80% or 85% or 90% identity to said sequence, and most preferably at least 95%, 96%, 97%, 98% or 99% identity to said amino acid sequence.

40. A composition for use in inhibiting hair production according to claim 38 or 39 wherein the variant comprises or consists of an amino acid sequence of SEQ ID NO:
69 or 70, or a fragment thereof, in which one or more amino acids is conservatively substituted.

41. A composition for use in inhibiting hair production according to any one of claims 38 to 40 wherein the polypeptide comprises or consists of one or more additional amino acids, inserted at the N- and/or C-terminus and/or internally within the amino acid sequence of SEQ ID NO:69 or 70.

42. A composition for use in inhibiting hair production according to claim 41 wherein the polypeptide comprises or consists of at least 2, 3, 4, 5, 6, 7, 8, 9, 10, 15 or 20 additional amino acids.

43. A composition for use in inhibiting hair production according to claim 42 wherein the additional amino acids are the amino acids from the corresponding positions of the wildtype murine osteopontin.

44. A composition for use in inhibiting hair production according to any one of claim 38 or 39 wherein the polypeptide comprises or consists of an amino acid sequence of SEQ ID NO: 134, or of a fragment or variant thereof
SEQ ID NO: 135
VDVPNGRGDSLAYGLR.

45. A composition for use in inhibiting hair production according to claim 44 wherein the fragment comprises 15 or fewer amino acids in length, for example 14, 13, 12, 11, 10, 9, 8, 7, 6 or 5 amino acids in length.

46. A composition for use in inhibiting hair production according to claim 44 wherein the polypeptide comprises or consists of a variant of the amino acid sequence of SEQ ID NO: 135, or of a fragment thereof.

47. A composition for use in inhibiting hair production according to claim 46 wherein the variant comprises or consists of an amino acid sequence with at least 50% identity to the amino acid sequence of SEQ ID NO: 135, more preferably at least 60%, 70% or 80% or 85% or 90% identity to said sequence, and most preferably at least 95%, 96%, 97%, 98% or 99% identity to said amino acid sequence.

48. A composition for use in inhibiting hair production according to claim 46 or 47 wherein the variant comprises or consists of an amino acid sequence of SEQ ID NO:135, or a fragment thereof, in which one or more amino acids is conservatively substituted.
A composition for use in inhibiting hair production according to claim 46 or 47 wherein the variant comprises or consists of an amino acid sequence of SEQ ID NO:135, or a fragment thereof, in which the RGD sequence is inactivated.

49. A composition for use in inhibiting hair production according to any one of claims 46 to 49 wherein the polypeptide comprises or consists of one or more additional amino acids, inserted at the N- and/or C-terminus and/or internally within the amino acid sequence of SEQ ID NO:135.

50. A composition for use in inhibiting hair production according to claim 49 wherein the polypeptide comprises or consists of at least 2, 3, 4, 5, 6, 7, 8, 9, 10, 15 or 20 additional amino acids.

51. A composition for use in inhibiting hair production according to claim 49 or 50 wherein the additional amino acids are the amino acids from the corresponding positions of the wildtype murine osteopontin.

52. A composition for use in inhibiting hair production according to any one of the preceding claims wherein the polypeptide is non-naturally occurring.

53. A composition for use in inhibiting hair production according to any one of the preceding claims wherein the polypeptide comprises or consists of tandem repeats.

54. A composition for use in inhibiting hair growth according to claim 53 wherein the tandem repeats comprise or consist of the amino acid sequence of any one or more of SEQ ID NOS: 1 to 65.

55. A composition for use in inhibiting hair growth according to claim 54 wherein the tandem repeats comprise or consist of the amino acid sequence of SEQ ID NO:1 or 26.

56. A composition for use in inhibiting hair growth according to claim 53 wherein the tandem repeats comprise or consist of the amino acid sequence of any one or more of SEQ ID NOS: 69 to 133.

57. A composition for use in inhibiting hair growth according to claim 55 wherein the tandem repeats comprise or consist of the amino acid sequence of SEQ ID NO: 69 and/or 94.

58. A composition for use in inhibiting hair production according to any one of the preceding claims wherein the polypeptide is modified or derivatised at one or more amino acid positions.

59. A composition for use in inhibiting hair production according to claim 58 wherein the polypeptide is glycosylated at one or more amino acid positions.

60. A composition for use in inhibiting hair production according to claim 58 wherein the polypeptide is PEGylated at one or more amino acid positions.

61. A composition for use in inhibiting hair production according to any one of the preceding claims for topical administration.

62. A composition for use in inhibiting hair production according to any one of the preceding claims for transdermal administration.

63. A composition for use in inhibiting hair production according to any one of the preceding claims for intracutaneous administration.

64. A composition for use in inhibiting hair production according to any one of the preceding claims wherein the mammal is a human.

65. A composition for use in inhibiting hair production according to any one of claims 1 to 64 for treating or preventing a disease or disorder associated with unwanted and/or excessive hair growth in a mammal.

66. A composition for use in inhibiting hair production according to claim 65 wherein the disease or disorder associated with unwanted and/or excessive hair growth is hirsutism.

67. A composition for use in inhibiting hair production according to claim 65 or 66 wherein the disease or disorder associated with unwanted and/or excessive hair growth is selected from the groups consisting of polycystic ovary syndrome (PCOS), the most common cause, congenital adrenal hyperplasia, Cushing's disease, growth hormone excess (acromegaly), tumours in the ovaries, adrenal gland cancer, Von Hippel-Lindau disease, insulin resistance, stromal hyperthecosis (SH), obesity, porphyria cutanea tarda, side effects of medication (such as tetrahydrogestrinone, phenytoin, minoxidil), hormonal treatment and hormonal disorders.

68. A composition for use in inhibiting hair production according to claim 65 for treating or preventing ingrown hair in a mammal.

69. A composition for use in inhibiting hair production according to claim 68 wherein the ingrown hair is associated with shaving, waxing and/or acne.

70. Use of a composition according to any one of claims 1 to 64 in the preparation of a medicament for treating or preventing a disease or disorder associated with unwanted and/or excessive hair production in a mammal in a mammal.

71. The use according to claim 70 wherein the disease or disorder associated with unwanted and/or excessive hair production is hirsutism.

72. The use according to claim 70 or 71 wherein the disease or disorder associated with unwanted and/or excessive hair production is selected from the groups consisting of polycystic ovary syndrome (PCOS), the most common cause, congenital adrenal hyperplasia, Cushing's disease, growth hormone excess (acromegaly), tumours in the ovaries, adrenal gland cancer, Von Hippel-Lindau disease, insulin resistance, stromal hyperthecosis (SH), obesity, porphyria cutanea tarda, side effects of medication (such as tetrahydrogestrinone, phenytoin, minoxidil), hormonal treatment and hormonal disorders.

73. The use according to claim 70 wherein the medicament is for treating or preventing ingrown hair in a mammal.

74. The use according to claim 73 wherein the ingrown hair is associated with shaving, waxing and/or acne.

75. A method for treating or preventing a disease or disorder associated with unwanted and/or excessive hair growth in a mammal, the method comprising administering to the mammal an effective amount of a composition according to any one of claims 1 to 64.

76. A method according to claim 75 wherein the disease or disorder associated with unwanted and/or excessive hair production is hirsutism.

77. A method according to claim 75 or 86 wherein the disease or disorder associated with unwanted and/or excessive hair production is selected from the groups consisting of polycystic ovary syndrome (PCOS), the most common cause, congenital adrenal hyperplasia, Cushing's disease, growth hormone excess (acromegaly), tumours in the ovaries, adrenal gland cancer, Von Hippel-Lindau disease, insulin resistance, stromal hyperthecosis (SH), obesity, porphyria cutanea tarda, side effects of medication (such as tetrahydrogestrinone, phenytoin, minoxidil), hormonal treatment and hormonal disorders.

78. A method according to claim 76 for treating or preventing ingrown hair in a mammal.

79. A method according to claim 78 wherein the ingrown hair is associated with shaving, waxing and/or acne.

80. A method according to any one of claims 75 to 79 wherein the mammal is human.

81. A method according to any one of claims 75 to 80 wherein the mammal is male.

82. A method according to any one of claims 75 to 80 wherein the mammal is female.

83. Use of a composition according to any one of claims 1 to 64 for cosmetic hair removal in a human.

84. The use according to claim 83 wherein the human is male.

85. The use according to claim 84 wherein the hair is removed from an area of the body selected from the group consisting of face (e.g. cheeks, chin and above the upper lip), chest, shoulders, neck and back.

86. The use according to claim 83 wherein the human is female.

87. The use according to claim 86 wherein the hair is removed from an area of the body selected from the group consisting of face (e.g. cheeks, chin and above the upper lip), back, legs, arms, fingers, feet, toes and pubis.

88. A method for cosmetic hair removal in a human comprising administering to the human an effective amount of a composition according to any one of claims 1 to 64.

89. A method according to claim 88 wherein the human is male.

90. A method according to claim 88 or 89 wherein the hair is removed from an area of the body selected from the group consisting of face (e.g. cheeks, chin and above the upper lip), chest, shoulders, neck and back.

91. A method according to claim 88 wherein the human is female.

92. A method according to claim 91 wherein the hair is removed from an area of the body selected from the group consisting of face (e.g. cheeks, chin and above the upper lip), back, legs, arms, fingers, feet, toes and pubis.

93. Use of a polypeptide as defined in any one of claims 1 to 64 ex vivo or in vitro for inhibiting hair production.

94. A use according to claim 93 for inhibiting hair growth on a skin explant prior to grafting of the explant on to a mammal.

95. A use according to claim 93 for inhibiting hair growth follicles (or stem cell precursors of the same) in vitro.

96. A composition for use in the inhibition of hair production in mammals substantially as defined herein with reference to the description and figures.

97. A use substantially as defined herein with reference to the description.

98. A method substantially as defined herein with reference to the description.
